(19) United States
(12) Patent Application Publication
(10) Pub. No.: US 2025/0345656 A1
(43) Pub. Date: Nov. 13, 2025

(54) GARMENTS AND METHODS FOR DETECTING ONE OR MORE CHARACTERISTICS OF A WEARER

(71) Applicant: Theo Health Limited, Glasgow (GB)

(72) Inventor: Jodie Sinclair, Glasgow (GB)

(73) Assignee: Theo Health Limited, Glasgow (GB)

(21) Appl. No.: 18/854,219

(22) PCT Filed: Apr. 6, 2023
(86) PCT No.: PCT/GB2023/050944

§ 371 Date: Oct. 4, 2024

(30) Foreign Application Priority Data
Apr. 8, 2022 (GB) 2205180.9

(51) Int. Cl. A63B 24/00; A41D 1/00

(52) U.S. Cl. CPC A63B 24/0062; A41D 1/002; A63B 24/0075; A41D 2400/00; A63B 2024/0068; A63B 2225/74; A63B 2230/605; A63B 2230/65; A63B 2230/70

(57) ABSTRACT

An apparatus (1) comprising a garment (2), the garment comprising: at least one muscle activity sensor (4, 34) for detecting activity of a muscle at least partially covered by the garment; at least one electrical visual indicator device (14); and a controller (11) configured to: receive an activity signal indicative of detected activity of the muscle from the at least one muscle activity sensor; and cause the at least one electrical visual indicator device to output a visual indication of the detected activity of the muscle in dependence on the received activity signal.

### FIELD OF THE INVENTION

The present inventions relate to garments and methods of using the same, each for detecting, determining and/or displaying one or more characteristics of a wearer.

### BACKGROUND TO THE INVENTION

Exercise is essential for maintaining a healthy lifestyle. However, there are many circumstances in which a person seeking to perform a particular exercise, might perform the exercise incorrectly. For example, the person might be unfamiliar with the exercise, or might find the exercise difficult and so perform it improperly in an attempt to make it easier. Alternatively, the person may be recovering from an injury, making it more challenging to correctly perform particular exercises. Unfortunately, the incorrect performance of exercises typically makes the exercises less effective in terms of health benefits. In some case, incorrect performance of exercises can also lead to injury.

The risk of incorrect performance of exercises can be reduced if the person performing the exercise has the assistance of a second person, for example a personal trainer or a physiotherapist. However, even in this case, it can be difficult for the second person to be certain of how well the exercise is being performed. For example, it can be difficult to tell whether the person performing the exercise is using muscles other than the intended muscles to help them to complete the exercise (which often happens where the intended muscles are less developed or injured).

It is in this context that the present disclosure has been devised.

### SUMMARY OF THE INVENTION

According to a first aspect of the invention, there is provided an apparatus comprising a garment and a controller, the garment comprising:
at least one muscle activity sensor for detecting activity of a muscle at least partially covered by the garment; and
at least one electrical visual indicator device,
wherein the is controller configured to:
   receive an activity signal indicative of detected activity of the muscle from the at least one muscle activity sensor; and
   cause the at least one electrical visual indicator device to output a visual indication of the detected activity of the muscle in dependence on the received activity signal.

The at least one muscle activity sensor may comprise at least two sensor electrodes configured to contact the skin of a wearer of the garment. The at least one muscle activity sensor may be configured to detect activity of a muscle. The at least one muscle activity sensor may comprise one or more reference electrodes. In an example, the at least one muscle activity sensor may comprise one reference electrode (e.g. exactly one reference electrode). One or more of the electrodes may be be (e.g. at least partially) flexible. One or more of the electrodes may be (e.g. at least partially) resiliently deformable. For example, one or more of the electrodes may be flexible enough to flex in response to movements of the wearer, preferably whilst still maintaining sufficient contact with the wearer that the activity signal is not interrupted. One or more of the electrodes may be resiliently deformable to resiliently deform in response to movements of the wearer, preferably whilst still maintaining sufficient contact with the wearer that the activity signal is not interrupted. The muscle activity sensor may comprise (e.g. be) an electromyography (EMG) sensor. The or each electrode may comprise (e.g. be) an EMG electrode. The activity signal may comprise (e.g. be) an EMG signal.

The controller may comprise one or more processors and a computer-readable memory (e.g. a non-transitory computer readable storage medium) storing instructions which, when executed by the one or more processors, cause the controller to perform the actions for which the controller is configured. The garment may comprise the controller.

Activity of a muscle may comprise movement of at least part of a muscle. Activity of a muscle may comprise contraction of at least part of a muscle. Activity of a muscle 8 may comprise relaxation of at least part of a muscle. Activity of a muscle may comprise a change in tension of at least part of a muscle.

The electrodes may be dry electrodes. The electrodes may be wet electrodes. The electrodes may be active electrodes. The electrodes may be passive electrodes.

The garment may comprise an inner surface configured to be in contact with the skin of the wearer when the garment is worn by the wearer. The garment may comprise an outer surface configured to be visible when the garment is worn. The one or more electrodes may be defined on (e.g. mounted on) the inner surface of the garment, e.g. such that the one or more electrodes are configured to be in contact with the skin of the wearer when the garment is worn by the wearer.

According to a further aspect of the invention there is provided a method of use of an apparatus comprising a garment, when the garment is worn by a wearer whilst performing an exercise, the garment comprising:
at least one muscle activity sensor for detecting activity of a muscle at least partially covered by the garment; and
at least one electrical visual indicator device,
the method comprising:
receiving an activity signal indicative of a detected activity of the muscle from the at least one muscle activity sensor; and
causing the at least one electrical visual indicator device to output a visual indication of the activity of the muscle in dependence on the received activity signal.

The provision of an apparatus or a method wherein a garment comprises muscle activity sensors to detect activity of a muscle, and an electrical visual indicator device outputs a visual indication of muscle activity, advantageously allows one or more users (e.g. a wearer, a personal trainer, or a healthcare professional such as a physiotherapist) to observe and assess muscle activity during exercise. This can be helpful in a variety of ways, including for example to allow a healthcare professional to observe whether and how a prescribed exercise is being performed. It also allows a wearer to observe whether they are activating an intended muscle and, for example, whether a muscle is (e.g. becoming) fatigued. Furthermore, the visual indication can be seen in a mirror, with the result that it is easier for a wearer of the garment to maintain good posture when completing an exercise. This is because the wearer does not need to obtain information about muscle activity from another device (e.g. a laptop or smartphone) which would alter their posture during the exercise.

The at least one electrical visual indicator device may be a plurality of electrical visual indicator devices. The at least one electrical visual indicator device may comprise one or more (e.g. a plurality of) light emitters. For example, the at least one electrical visual indicator device may comprise one or more bulbs. The plurality of light emitters may comprise one or more groups of light emitters. The at least one electrical visual indicator device may comprise one or more light emitting diodes (LEDs). The at least one electrical visual indicator device may comprise a plurality of LEDs. The plurality of LEDs may comprise one or more groups of LEDs. The or each LED may be a flexible LED. The or each LED may be an LED thread. The at least one electrical visual indicator device may comprise (e.g. be) one or more electrophoretic visual indicator devices (e.g. displays), for example one or more electronic paper visual indicator devices or electronic ink (e-ink) visual indicator devices.

The controller may be configured to cause one or more of the plurality of light emitters to illuminate (e.g. output light) in dependence on the received activity signal. The controller may be configured to cause one or more of the plurality of LEDs to illuminate (e.g. output light) in dependence on the received activity signal.

Causing the at least one electrical visual indicator device to output a visual indication of the activity of the muscle may comprise causing one or more of the plurality of light emitters to illuminate (e.g. output light) in dependence on detected activity of the muscle. Causing the at least one electrical visual indicator device to output a visual indication of the activity of the muscle may comprise causing one or more of the plurality of LEDs to illuminate (e.g. output light) in dependence on detected activity of the muscle.

Electrical visual indicator devices comprising lights, and particularly LEDs, are particularly convenient and efficient. Furthermore, a user of the method or apparatus will straightforwardly understand how the method or apparatus works, when they observe the lights or LEDs illuminating (e.g. outputting light) in response to their movement (and thus their muscle activation).

The apparatus may comprise a plurality of muscle activity sensors. The at least one muscle activity sensor may comprise (e.g. be) a plurality of muscle activity sensors. Each muscle activity sensor may be configured to detect activity of a muscle at least partially covered by the garment, in a respective region of the body of the wearer. For example, a first muscle activity sensor may be configured to detect activity of a first muscle in a first respective region of the body of the wearer, and that first respective region may be a different respective region to the respective region for which each other muscle activity sensor is configured to detect activity of a muscle. However, in some examples, it may be the case that more than one muscle activity sensor is configured to detect activity of a muscle in the same respective region. Each muscle activity sensor may be configured to detect muscle activity of a respective muscle group (optionally a different respective muscle group to each other muscle activity sensor). Each muscle activity sensor may be configured to detect muscle activity of a respective muscle (optionally a different respective muscle to each other muscle activity sensor).

Where a plurality of muscle activity sensors are provided, this allows for observation of activity in multiple muscles, or multiple regions, or multiple muscle groups. This is particularly helpful for allowing a comparison between the activation of muscles in different limbs, for example. It is also helpful for observing a sequence of muscle activations as different muscles are activated during an exercise.

The garment may be configured such that the at least one electrical visual indicator device is arranged to be (e.g. positioned) at least partially over a muscle for which activity is to be detected by the at least one muscle activity sensor, e.g. when the garment is worn. For example, the garment may comprise a muscle activity sensor arranged to detect activity of a particular muscle (e.g. a quadricep) and at least one electrical visual indicator device may be arranged to be (e.g. positioned) at least partially over the particular muscle (e.g. the quadricep), e.g. when the garment is worn. The garment may comprise a muscle activity sensor arranged to detect activity of a particular muscle group (e.g. one or more abdominal muscles) and at least one electrical visual indicator device may be arranged to be (e.g. positioned) at least partially over the particular muscle group (e.g. the one or more abdominal muscles), e.g. when the garment is worn. It may be that a location of the or each of the at least one electrical visual indicator device is arranged to be over the muscle or muscle group for which activity of the muscle is detected to cause output of the visual indication by the or the respective at least one electrical visual indicator device. In other words, the user of the apparatus can easily tell which muscle is being activated because the visual indication is output from a location on the garment which corresponds to the location of the muscle in the wearer.

Although the processor may be configured to receive a combined electrical indication of detected activity of one or muscles from the plurality of muscle activity sensors, this is not required. In some embodiments, the controller may be configured to receive a respective activity signal indicative of detected activity of a muscle from each respective muscle activity sensor. The controller may be configured to cause the at least one electrical visual indicator device to output a visual indication of the detected activity of the muscle in the respective region of the body of the wearer, in dependence on each (e.g. respective) activity signal. The controller may be configured to cause the at least one electrical visual indicator device to output a visual indication of the detected activity of one or more muscles, in dependence on each activity signal (optionally in dependence on a combination of one or more activity signals). The method may comprise receiving a respective activity signal indicative of detected activity of a muscle from each muscle activity sensor. The method may comprise causing the at least one electrical visual indicator device to output a visual indication of the detected activity of the muscle in the respective region of the body of the wearer, in dependence on each activity signal. The method may comprise causing the at least one electrical visual indicator device to output a visual indication of the detected activity of one or more muscles, in dependence on each activity signal (optionally in dependence on a combination of one or more activity signals).

Accordingly a user of the apparatus, or a wearer of the garment, can advantageously observe the activation of different muscles (or muscle groups). For example, if a wearer performs a stiff leg deadlift, and one of the muscle activity sensors is arranged to detect activation of a hamstring muscle, the electrical visual indicator device will output an indication of muscle activation of the hamstring muscle. This will help a user or wearer to know that the hamstring muscle has been activated as intended (rather than, for example, the wearer performing the exercise incorrectly and activating some other muscles, such as those in the lower back).

The at least one electrical visual indicator device may comprise a plurality of electrical visual indicator devices. The plurality of electrical visual indicator devices may be arranged in one or more groups. The plurality of electrical visual indicator devices may be arranged in a pattern. The plurality of electrical visual indicator devices may be arranged in one or more lines. The plurality of electrical visual indicator devices may be arranged in one or more lines, to thereby have the appearance of muscle fibres. For example, the plurality of light emitters (e.g. LEDs) may be arranged in one or more groups. The plurality of light emitters (e.g. LEDs) may be arranged in a pattern. The plurality of light emitters (e.g. LEDs) may be arranged in one or more lines. The plurality of light emitters (e.g. LEDs) may be arranged in a plurality of lines. The plurality of light emitters (e.g. LEDs) may be arranged in one or more lines to thereby have the appearance of muscle fibres.

This provides the advantage that the controller or the method can cause the LEDs to illuminate (e.g. to output light) in sequence as the wearer of the garment performs exercises. This is particularly helpful to wearers who are performing exercises alone, as it provides visual feedback as to how well an exercise is being performed and this feedback can be understood intuitively. For example, if the wearer is performing a squat, they can observe that they have activated the muscles required to perform the descending part of the squat (because the lines of LEDs representing the fibres of these muscles will illuminate), and subsequently the muscles required to perform the ascending part of the squat. This could help the wearer to understand that the descending part of the squat should be extended further, for example, or that they are not properly activating the muscles in one of their legs.

The controller may be configured to cause (or the method may comprise causing) a plurality of electrical visual indicator devices (e.g. the plurality of light emitters, optionally the plurality of LEDs) to output a first visual indication of the detected activity of the muscle during a first part of an exercise in dependence on the received activity signal during the first part of the exercise. The controller may be configured to cause (or the method may comprise causing) a plurality of electrical visual indicator devices (e.g. the plurality of light emitters, optionally the plurality of LEDs) to (e.g. subsequently) output a second visual indication of the detected activity of the muscle during a second part of an exercise in dependence on the received activity signal during the second part of the exercise. For example, the controller may be configured to cause (or the method may comprise causing) a first electrical visual indicator device, optionally a first light emitter or first group of light emitters, e.g. a first LED or first group of LEDs (of the said plurality) to output a first visual indication during a first part of an exercise and a second electrical visual indicator device optionally a second light emitter or second group of light emitters, e.g. a second LED or second group of LEDs (of the said plurality) to output a second visual indication during a second part of the exercise and optionally one or more further electrical visual indicator devices optionally a further light emitter or further group of light emitters, e.g. a further LED or further group of LEDs (of the said plurality) to output one or more respective further visual during one or more further parts of the exercise.

The visual indication may comprise at least two visual indication modes. Each visual indication mode may be a different visual indication mode to each other visual indication mode (e.g. of the at least two visual indication modes). The at least one electrical visual indicator device may be configured to output visual indications in the at least two visual indication modes. The controller may be configured to cause the at least one electrical visual indicator device to output a visual indication in one of the at least two visual indication modes, in dependence on the received activity signal. The controller may be configured to cause the at least one electrical visual indicator device to output visual indications in a respective visual indication mode in dependence on received activity signal. The method may comprise causing the at least one electrical visual indicator device to output a visual indication in one of the at least two visual indication modes, in dependence on the received activity signal.

The at least one muscle activity sensor may comprise at least one quadricep muscle activity sensor. The at least one muscle activity sensor may be arranged to be positioned at least partially over a quadricep of the wearer, e.g. when the garment is worn. The at least one muscle activity sensor may comprise at least one bicep muscle activity sensor. The at least one muscle activity sensor may be arranged to be positioned at least partially over a bicep of the wearer, e.g. when the garment is worn. The at least one muscle activity sensor may comprise at least one triceps muscle activity sensor. The at least one muscle activity sensor may be arranged to be positioned at least partially over a triceps of the wearer, e.g. when the garment is worn. The at least one muscle activity sensor may comprise at least one deltoid muscle activity sensor. The at least one muscle activity sensor may be arranged to be positioned at least partially over a deltoid of the wearer, e.g. when the garment is worn. The at least one muscle activity sensor may comprise at least one pectoral muscle activity sensor. The at least one muscle activity sensor may be arranged to be positioned at least partially over a pectoral muscle of the wearer, e.g. when the garment is worn. The at least one muscle activity sensor may comprise at least one abdominal muscle activity sensor. The at least one muscle activity sensor may be arranged to be positioned at least partially over an abdominal muscle of the wearer, e.g. when the garment is worn. The at least one muscle activity sensor may comprise at least one gluteus maximus muscle activity sensor. The at least one muscle activity sensor may be arranged to be positioned at least partially over a gluteus maximus of the wearer, e.g. when the garment is worn.

For example, the controller may be configured to cause the at least one electrical visual indicator device to output:
a visual indication in a first visual indication mode if the activity signal is indicative of a first degree of activity of the muscle; and
a visual indication in a second visual indication mode, different to the first visual indication mode, if the activity signal is indicative of a second degree of activity 17 of the muscle.

The controller may be configured to cause the at least one electrical visual indicator device to output a visual indication in one or more further visual indication modes. For example, the controller may be configured to cause the at least one electrical visual indicator device to output a visual indication in a third visual indication mode if activity signal is not indicative of activity of a muscle.

The method may comprise outputting:
a visual indication in a first visual indication mode if the activity signal is indicative of a first degree of activity of the muscle; and
a visual indication in a second visual indication mode, different to the first visual indication mode, if the activity signal is indicative of a second degree of activity of the muscle.

The method may comprise outputting a visual indication in one or more further visual indication modes. For example, the method may comprise outputting a visual 34 indication in a third visual indication mode if the activity signal is not indicative of activity of a muscle.

The controller may be configured to determine (or the method may comprise determining) a maximum activity of the muscle (e.g. in dependence on the activity signal when the wearer indicates that they are activating the muscle as much as they are able). The controller may be configured to determine (or the method may comprise determining) a rest activity of the muscle (e.g. in dependence on the activity signal when the muscle is not being activated, optionally when the muscle and/or the wearer is at rest).

The controller may be configured to determine (or the method may comprise determining) one or more muscle activity zones in dependence on the maximum activity of the muscle. The controller may be configured to determine (or the method may comprise determining) one or more muscle activity zones (e.g. further) in dependence on the rest activity of the muscle. The first degree of activity may be a first muscle activity zone. The second degree of activity may be a second muscle activity zone.

The one or more muscle activity zones may comprise a resting muscle activity zone (e.g. at the rest activity, optionally up to 5% above the rest activity, optionally up to 10% above the rest activity, optionally up to 20% above the rest activity, for example where these percentages are determined as a percentage of the amplitude of the activity signal of the maximum activity of the muscle). The one or more muscle activity zones may comprise a maximum muscle activity (e.g. at the maximum activity, optionally up to 95% of the maximum activity, optionally up to 90% of the maximum activity, optionally up to 80% of the maximum activity, for example where these percentages are determined as a percentage of the amplitude of the activity signal of the maximum activity of the muscle). The one or more muscle activity zones may comprise one or more intermediate muscle activity zones. For example, the one or more muscle activity zones may comprise one or more of: a zone indicative of the muscle being activated by from 0% to 20% of the maximum activity; a zone indicative of the muscle being activated by from 21% to 40% of the maximum activity; a zone indicative of the muscle being activated by from 41% to 60% of the maximum activity; a zone indicative of the muscle being activated by from 61% to 80% of the maximum activity; and/or a zone indicative of the muscle being activated by from 81% to 100% (optionally more than 100%) of the maximum activity (for example where these percentages are determined as a percentage of the amplitude of the activity signal of the maximum activity of the muscle).

The controller may be configured to output (or the method may comprise outputting) the one or more muscle activity zones. For example, the controller may be configured to output (or the method may comprise outputting) the one or more muscle activity zones to a data store (e.g. a database). The controller may be configured to output (or the method may comprise outputting) the one or more muscle activity zones to a (e.g. the) application. The apparatus may be configured to cause (or the method may comprise causing) the at least one visual indicator to output a visual indication of the one or more muscle activity zones.

Causing the at least one electrical visual indicator device to output a visual indication may comprise illuminating the at least one electrical visual indicator device. The controller may be configured to cause (or the method may comprise causing) a plurality of electrical visual indicator devices (optionally a plurality of LEDs) to output a visual indication of the detected activity of the muscle in dependence on the received activity signal. Causing a plurality of electrical visual indictor devices (optionally a plurality of LEDs) to output a visual indication may comprise illuminating one or more of the electrical visual indicator devices of the said plurality. The at least one visual indicator device may emit light of a first colour (e.g. wavelength) and causing the at least one electrical visual indicator device to output a visual indication may comprise causing the at least one electrical visual indictor device to emit light of a second colour (e.g. wavelength) different to the first colour (e.g. wavelength). One or more of the plurality of electrical visual indicator devices (optionally a plurality of LEDs) may emit light of a first colour (e.g. wavelength) and causing the at least one electrical visual indicator device to output a visual indication may comprise causing at least one of the plurality of electrical visual indicator devices (optionally a plurality of LEDs) to emit light of a second colour (e.g. wavelength) different to the first colour (e.g. wavelength). Causing the at least one electrical visual indicator device to output a visual indication may comprise illuminating the at least one electrical visual indicator device and then causing the electrical visual indicator device to stop illuminating, and then repeating this. Causing the plurality of electrical visual indicator devices (optionally a plurality of LEDs) to output a visual indication may comprise illuminating at least one electrical visual indicator device of the plurality of visual indicator devices, and then causing the said electrical visual indicator device to stop illuminating, and then repeating this. The at least one visual indicator device may emit light of a first brightness, and causing the at least one electrical visual indicator device to output a visual indication may comprise causing the at least one electrical visual indictor device to emit light of a second brightness different to the first colour. One or more of the plurality of electrical visual indicator devices (optionally a plurality of LEDs) may emit light of a first brightness, and causing the at least one electrical visual indicator device to output a visual indication may comprise causing at least one of the plurality of electrical visual indicator devices (optionally a plurality of LEDs) to emit light of a second brightness different to the first brightness. The first brightness may be zero brightness (e.g. the electrical visual indicator may be off or may be emitting substantially no light). The second brightness may be zero brightness (e.g. the electrical visual indicator may be off or may be emitting substantially no light). There may be third, fourth or further different brightnesses. There may be third, fourth, or further different colours (e.g. wavelengths).

The controller may be configured to cause (or the method may comprise causing) a (e.g. plurality of) electrical visual indicator device(s) (optionally a plurality of LEDs) to output a visual indication of the detected activity of the muscle in dependence on the received activity signal. Causing a (e.g. plurality of) electrical visual indicator device(s) (optionally a plurality of LEDs) to output a visual indication may comprise causing a first electrical visual indicator device (e.g. first LED, optionally first group of LEDs) (of the said plurality) to output a visual indication, and subsequently causing one or more further visual indicator devices (e.g. further LEDs, optionally further groups of LEDs) (of the said plurality) to output a visual indication. For example, causing a (e.g. plurality of) electrical visual indicator device(s) to output a visual indication may comprise illuminating a first electrical visual indicator device (e.g. first LED, optionally first group of LEDs), and then illuminating a second electrical visual indicator device (e.g. second LED, optionally second group of LEDs), and optionally then illuminating a third electrical visual indicator device (e.g. third LED, optionally third group of LEDs). The second electrical visual indicator device (e.g. LED, optionally group of LEDs) may be adjacent to the first electrical visual indicator device (e.g. LED or group of LEDs). The third electrical visual indicator device (e.g. LED, optionally group of LEDs) may be adjacent to the first and/or second electrical visual indicator device (e.g. LED, optionally group of LEDs). Accordingly, causing a plurality of electrical visual indicator devices (e.g. LEDs) to output a visual indication may comprise causing outputting a visual indication comprising a sequence visual indications.

Thus, different visual indication modes can be used to easily distinguish between different degrees of activity of the muscle.

The first degree of activity may be a greater degree of activity than the second degree of activity. The second degree of activity may be a greater degree of activity than the first degree of activity. For example, the first degree of activity may be indicative of the muscle being activated more intensely (for example because the muscle is being further contracted) than the second degree of activity.

Outputting a visual indication in a first visual indication mode may comprise causing the at least one electrical visual indicator device to output a visual indication having a different appearance to that which is output in a second visual indication mode. For example, outputting a visual indication in a first visual indication mode may comprise causing a light emitter to emit light of a first intensity, and outputting a visual indication in a second visual indication mode may comprise causing a light emitter to emit light of a second intensity different to the first intensity. Alternatively or additionally, outputting a visual indication in a first visual indication mode may comprise causing a light emitter to emit light of a first colour (e.g. wavelength or range of wavelengths), and outputting a visual indication in a second visual indication mode may comprise causing a light emitter to emit light of a second colour (e.g. wavelength or range of wavelengths) different to the first colour (e.g. wavelength or range of wavelengths). The skilled person will appreciate that there are many ways of causing visual indicators to output visual indications of different types, and thus many ways of arriving at different visual indication modes, each within the scope of the invention.

In an example, outputting a visual indication may comprise causing a first light emitter (e.g. LED) to illuminate and subsequently causing a second light emitter (e.g. LED) to illuminate as the first light emitter stops illuminating. Where a plurality of light emitters are arranged in a pattern (e.g. in a series of lines), outputting a visual indication may comprise causing one or more first light emitters (e.g. LEDs) to illuminate and subsequently causing one or more second light emitters (e.g. LEDs) to illuminate as the first light emitter(s) stops illuminating, and optionally further subsequently causing one or more third light emitters (e.g. LEDs) to illuminate as the second light emitter(s) stop illuminating. The second light emitter(s) may be (e.g. laterally) between the first and third light emitter(s). In this way a “wave effect” can be achieved, with light emitters illuminating in sequence.

Muscles can be activated to a lesser or greater degree. For example, the degree of muscle activation required to lift 1 kilogram is less than that required to lift 20 kilograms. Accordingly, by providing different visual indication modes for different degrees of muscle activation, a user or wearer can determine to what degree a muscle is being activated, and thus how hard that muscle is working. This is particularly helpful for physiotherapy. For example, after an injury it might be more difficult for an injured person to use a muscle and the injured person might therefore activate other muscles or muscle groups in order to support that muscle. In this case, the electronic visual indicator devices might output a visual indication that one muscle which should be activated during a particular exercise is only being activated to a first (e.g. lower) degree of activity, whilst other muscles which should not be activated are being activated to a second (e.g. higher) degree of activity. If unchecked, exercises performed in this way can lead to further injury. By providing such a visual indication, a user such as a physiotherapist has the information they need to know that they should either correct the performance of the exercise, or suggest a different exercise. Similarly, the wearer can monitor whether they are performing exercises correctly.

The controller may be configured to determine a frequency with which the activity signal is indicative of activation of a muscle. The controller may be configured to cause the at least one electrical visual indicator device to output a visual indication in a lower pace visual indication mode if the frequency is below (e.g. more than 5% below, e.g. more than 8% below, e.g. more than 10% below) a predetermined threshold frequency. The controller may be configured to cause the at least one electrical visual indicator device to output a visual indication in an upper pace visual indication mode if the frequency is above (e.g. more than 5% above, e.g. more than 8% above, e.g. more than 10% above) a predetermined threshold frequency. The controller may be configured to cause the at least one electrical visual indicator device to output a visual indication in a normal pace visual indication mode if the frequency is no more than 10%, for example no more than 8%, e.g. no more than 5% above or below the predetermined threshold frequency.

The method may comprise determining a frequency with which the activity signal is indicative of activation of a muscle. The method may comprise causing the at least one electrical visual indicator device to output a visual indication in a lower pace indication mode if the frequency is below (e.g. more than 5% below, e.g. more than 8% below, e.g. more than 10% below) a predetermined threshold frequency. The method may comprise causing the at least one electrical visual indicator device to output a visual indication in an upper pace visual indication mode if the frequency is above (e.g. more than 5% above, e.g. more than 8% above, e.g. more than 10% above) a predetermined threshold frequency. The method may comprise causing the at least one visual indicator device to output a visual indication in a normal pace visual indication mode if the frequency is no more than 10%, for example no more than 8%, e.g. no more than 5% above or below the predetermined threshold frequency.

In an example, outputting a lower pace visual indication may comprise causing a light emitter to emit light of a first (e.g. lower) intensity different to a second (e.g. higher) intensity that may be emitted when outputting an upper pace visual indication. Outputting a normal pace visual indication may comprise causing a light emitter to emit light of an intensity different to (optionally between) the first (e.g. lower) intensity of the light emitted for the lower pace visual indication and the second (e.g. higher) intensity of the light emitted for the higher pace visual indication. Alternatively or additionally, outputting a lower pace visual indication may comprise causing a light emitter to emit light of a first colour (e.g. wavelength or range of wavelengths) different to a second colour (e.g. wavelength or range of wavelengths) that may be emitted when outputting an upper pace visual indication. Outputting a normal pace visual indication may comprise causing a light emitter to emit light of a third colour (e.g. wavelength or range of wavelengths) different to (optionally between) the first colour (e.g. wavelength or range of wavelengths) of the light emitted for the lower pace visual indication and the second colour (e.g. wavelength or range of wavelengths) of the light emitted for the higher pace visual indication.

Many exercises take the form of repetitions (reps) of a particular movement or sequence of movements. For example, a person may carry out three sets of ten reps of a particular exercise. As well as the total number of reps carried out, the pace at which the reps are carried out is important. If the pace is too fast or too slow, this can lead to injury, or to the exercise being less effective than would otherwise be the case. Accordingly, by determining the frequency with which a muscle is activated, it is possible to determine the pace at which reps are carried out. If the pace is too high or too low, a visual indication can be output which will indicate to the user or wearer that they should adjust their pace accordingly, thereby reducing the risk of injury.

The apparatus may comprise a computer readable memory (e.g. a non-transitory computer readable storage medium). The computer readable memory may comprise an exercise data store (e.g. an exercise database). The computer readable memory (e.g. the exercise data store) may comprise exercise data (e.g. information about one or more exercises). The exercise information may comprise expected muscle activation information. For example, the exercise information may comprise information indicative of which muscles should be activated during a respective exercise. The exercise information may comprise expected exercise duration. The exercise information may comprise expected muscle activation frequency information. The exercise information may comprise expected pace information.

The controller may be configured to receive exercise information from the computer readable memory. The exercise information may be received in response to a request to receive the exercise information from the computer readable memory. The controller may be configured to determine when the wearer is performing an exercise in dependence on the received exercise information. The controller may be configured to determine when the wearer is performing an exercise (e.g. further) in dependence on the activity signal. The controller may be configured to determine when the wearer has completed the exercise in dependence on the received exercise information and the activity signal. The controller may be configured to cause the at least one electrical visual indicator device to output a rest phase visual indication when the exercise has been completed. The controller may be configured to cause the at least one electrical visual indicator device to stop outputting the rest phase visual indication after a predetermined period (e.g. subsequently causing the at least one electrical visual indicator device to output a visual indication when the exercise has been completed).

The method may comprise receiving exercise information from the computer readable memory. The exercise information may be received in response to a request to receive the exercise information from the computer readable memory. The method may comprise determining when the wearer is performing an exercise in dependence on the received exercise information. The method may comprise determining when the wearer is performing the exercise (e.g. further) in dependence on the activity signal. The method may comprise determining when the wearer has completed the exercise in dependence on the received exercise information and the activity signal. The method may comprise outputting a rest phase indication when the exercise has been completed, for example, the method may comprise causing the at least one electrical visual indicator device to output a rest phase visual indication when the exercise has been completed. Alternatively or additionally, the rest phase indication may be output via a haptic feedback device, or via a (e.g. the) application running on a device such as a laptop, smartphone, tablet, or smartwatch. The method may comprise outputting an indication that the rest phase has ended after a predetermined period, for example, the method may comprise causing the at least one electrical visual indicator device to stop outputting the rest phase visual indication after a predetermined period (e.g. subsequently causing the at least one electrical visual indicator device to output a visual indication when the exercise has been completed). Alternatively or additionally, the indication that the rest phase has ended may be output via a haptic feedback device, or via a (e.g. the) application running on a device such as a laptop, smartphone, tablet, or smartwatch.

Accordingly, this provides the user or wearer with a visual indication that an exercise (for example a rep, a set of reps, an exercise routine, etc.) has been completed. Many exercises should be followed by a rest period or a cooldown period. Without a rest or cooldown period, there is an increased risk of injury or discomfort. Providing a first indication that an exercise has been completed (e.g. by outputting a rest phase visual indication), and a second indication (e.g. stopping the rest phase visual indication) after a predetermined period, is a convenient way of indicating to a user or wearer that a sufficient rest period has passed. This limits the risk of the user moving on to further exercises too soon, for example.

In an example, outputting a rest phase visual indication may comprise causing a light emitter to emit light of a different (e.g. lower) intensity than the intensity of the light emitted during an exercise. Alternatively or additionally, outputting a rest phase visual indication may comprise causing a light emitter to emit light of a different colour (e.g. wavelength) to that of the light emitted during an exercise.

The apparatus may comprise a wired communication link. The apparatus may comprise a wireless communication link. The apparatus may comprise a computer readable storage medium. The apparatus may be configured to transmit and/or receive data (e.g. information) via a wired communication link. The method may comprise transmitting and/or receiving data (e.g. information) via a wired communication link. The apparatus may be configured to transmit and/or receive data (e.g. information) via a wireless communication link. The method may comprise transmitting and/or receiving data (e.g. information) via a wireless communication link. The apparatus may be configured to transmit and/or receive data (e.g. information) via Wi-Fi™. The method may comprise transmitting and/or receiving data (e.g. information) via Wi-Fi™. Wi-Fi™ is a family of wireless network protocols, based on the IEEE 802.11 family of standards. The apparatus may be configured to transmit and/or receive data (e.g. information) via Bluetooth™, optionally via Bluetooth low energy (Bluetooth 4.0 or Bluetooth LE). The method may comprise transmitting and/or receiving data (e.g. information) via Bluetooth™, optionally via Bluetooth low energy (Bluetooth 4.0 or Bluetooth LE). Bluetooth™ is a short-range wireless technology standard for transmitting data packets within the 2.4 GHz band.

The processor may be configured to determine whether the apparatus is connected to a wireless communication link. The processor may be configured to cause the at 9 least one electrical visual indicator device to output an indication of wireless connection in dependence on a determination of whether the apparatus is connected to a wireless communication link.

The apparatus may comprise at least one impedance sensor for taking a measurement indicative of a bioelectrical impedance of the wearer. The apparatus may comprise at least one impedance sensor configured to take a measurement indicative of bioelectrical impedance of the wearer. The apparatus or the garment may comprise at least one impedance sensor for detecting (e.g. configured to detect) a parameter indicative of a bioelectrical impedance of the wearer. The at least one impedance sensor typically comprises at least two electrodes. Alternatively or additionally, the apparatus may comprise a separate device which provides the impedance sensor. For example, the apparatus may comprise a smart scale.

The controller may be configured to receive the detected parameter. The controller may be configured to determine a body fat indicator indicative of an amount (e.g. a proportion) of body fat of the wearer (e.g. as a proportion of a total weight of the wearer or in relation to the proportion of the wearer not made up of body fat) in dependence on the detected parameter. The body fat indicator may be an estimate of body fat percentage of the wearer. The controller may be configured to receive the body fat indicator (e.g. an estimate of body fat percentage of the wearer). The controller may be configured to output the body fat indicator (e.g. the estimate of body fat percentage).

The method may comprise detecting a parameter indicative of a bioelectrical impedance of the wearer. The method may comprise receiving (e.g. from the impedance sensor) the detected parameter indicative of the bioelectrical impedance of the wearer. The method may comprise determining a body fat indicator indicative of an amount (e.g. a proportion) of body fat of the wearer (e.g. as a proportion of the total weight of the wearer or in relation to the proportion of the wearer not made up of body fat) in dependence on the parameter (indicative of the bioelectrical impedance of the wearer (e.g. the detected or received parameter)). The body fat indicator may be an estimate of the body fat percentage of the wearer. The method may comprise outputting the measurement of bioelectrical impedance. The method may comprise receiving a (e.g. the) body fat indicator. The method may comprise receiving an (e.g. the) estimate of body fat percentage of the wearer.

The at least two electrodes of the impedance sensor may comprise (e.g. be) at least two electrodes of the at least one muscle activity sensor. However, this is not required, and the at least two electrodes may comprise (e.g. be) at least two additional electrodes. Where the at least two electrodes of the impedance sensor comprise at least two electrodes of the muscle activity sensor, the impedance measurements and the muscle activity measurements are not typically carried out simultaneously.

The determination of the body fat indictor (e.g. the estimate of body fat percentage) described hereinbefore is itself believed to be novel and so, in a further aspect of the invention, there is provided an apparatus comprising a garment, the apparatus further comprising:
at least one impedance sensor for detecting a parameter indicative of a bioelectrical impedance of a wearer of the garment, the at least one impedance sensor comprising at least two electrodes;
the apparatus comprising a controller configured to:
receive the detected parameter; and
determine a body fat indicator indicative of an amount (e.g. a proportion) of body fat of the wearer (e.g. as a proportion of a total weight of the wearer or in relation to the proportion of the wearer not made up of body fat) in dependence on the detected parameter. The body fat indicator may be an estimate of the body fat percentage of the wearer. The controller may be configured to output body fat indicator (e.g. the estimate of body fat percentage). The garment may comprise the impedance sensor.

The further aspect of the present invention extends to a method of use of apparatus comprising a garment, wherein the garment is worn by a wearer. The apparatus further comprises at least one impedance sensor for detecting a parameter indictive of a bioelectrical impedance of a wearer of the garment, the at least one impedance sensor comprising at least two electrodes. The method comprises: receiving, from the impedance sensor, a parameter indicative of a bioelectrical impedance of the wearer; and determining a body fat indicator indicative of an amount (e.g. a proportion) of body fat of the wearer (e.g. as a proportion of a total weight of the wearer or in relation to the proportion of the wearer not made up of body fat) in dependence on the received parameter. The body fat indicator may be an estimate of the body fat percentage of the wearer.

The garment may comprise the at least one impedance sensor. The impedance sensor may be configured to take a measurement indicative of bioelectrical impedance of the wearer.

The amount (e.g. the proportion) of body fat of a person (e.g. as a proportion of the total weight of the person) and optionally the person's body fat percentage may be used as an indicator of health, and thus can be a helpful metric for a person to observe if they are seeking to improve their health. Furthermore, the proportion of body fat can be used to determine other parameters during exercise. For example, if the total weight and height of a wearer is known and their body fat percentage is known, this can be used to estimate the non-fat weight of the user's limbs. Furthermore, this information can be used (in combination with other parameters) to estimate the centre of gravity of the wearer. In addition, body fat proportion (e.g. percentage) measurements taken with impedance sensors are more typically carried out with an additional piece of equipment. Where a garment is provided comprising an impedance sensor, a user or wearer does not need such an additional piece of equipment.

The controller may be configured to adjust (and the method may comprise adjusting) the operation of one or more of the electrodes (optionally the electrodes of the muscle activity sensor) in dependence on the measurement indicative of bioelectrical impedance of the wearer. For example, the controller may be configured to alter (and the method may comprise altering) the amplitude applied by one or more of the electrodes (optionally the electrodes of the muscle activity sensor) in dependence on the measurement indicative of bioelectrical impedance of the wearer. The controller may be configured to alter (and the method may comprise altering) the current applied by one or more of the electrodes (optionally the electrodes of the muscle activity sensor) in dependence on the measurement indicative of bioelectrical impedance of the wearer. This is helpful, because the bioelectrical impedance of the wearer can affect the accuracy of the activity signal. As such, where a wearer has a higher-than-expected bioelectrical impedance, it can be useful to measure this and then adjust the operation of an electrode accordingly, to thereby achieve a more accurate activity signal.

The apparatus may comprise a computer readable memory (e.g. a non-transitory computer readable storage medium). The computer readable memory may comprise a biometric data store (e.g. a biometric database). The computer readable memory (e.g. the biometric data store) may comprise biometric data (e.g. biometric information). For example, the biometric data store may comprise height data indicative of the wearer's height. The biometric data store may comprise weight data indicative of the wearer's weight. The biometric data store may comprise height data indicative of the wearer's height. The biometric data store may comprise age data indicative of the wearer's age. The biometric data store may comprise gender data indicative of the wearer's gender. The biometric data store may comprise population data indicative of one or more biometric statistics of a population of individuals to which the wearer belongs. The processor may be configured to determine a body fat indicator indicative of a proportion of body fat of the wearer (e.g. as a proportion of a total weight of the wearer or in relation to the proportion of the wearer not made up of body fat) (e.g. further) in dependence on the biometric data. The body fat indicator may be an estimate of body fat percentage.

The apparatus may comprise a computer readable memory (e.g. a non-transitory computer readable storage medium) comprising an apparatus data store (e.g. an apparatus database), the apparatus data store (e.g. database) may comprise the biometric data store (e.g. database). The apparatus data store (e.g. database) may comprise the exercise data store (e.g. database).

The garment may comprise at least two first region electrodes arranged to contact the skin of a first region of a wearer of the garment and to thereby detect muscle activity in the first region. The garment may comprise at least two second region electrodes arranged to contact the skin of a second region of the wearer of the garment to thereby detect muscle activity in the second region. It will be understood that the first region is different to the second region.

The first region may be separated from the second region by a separation distance of at least 1 centimetre, optionally at least 2 centimetres, e.g. at least 5 centimetres. The first region may be up to 100 centimetres from the second region, optionally up to 50 centimetres, optionally up to 20 centimetres. The first region may be a region (e.g. at least partially) over a first muscle. The first region may be a region (e.g. at least partially) over a second muscle. The first region may be a region (e.g. at least partially) over a first muscle group. The first region may be a region (e.g. at least partially) over a second muscle group. For example, the first region may be at least partially over a right quadricep, and the second region may be at least partially over a left quadricep. The first region may be (e.g. at least partially) over an abdominal muscle and the second region may be (e.g. at least partially) over a dorsal muscle. The first region may be (e.g. at least partially) over a first muscle that is an opposing muscle to a second muscle (e.g. the first muscle may form an antagonistic pair with the second muscle) and the second region may be (e.g. at least partially) over the second muscle. The first region may be (e.g. at least partially) over a first muscle that is a complimentary muscle to a second muscle and the second region may be (e.g. at least partially) over the second muscle.

The first electrodes may be right-side electrodes arranged to contact the skin of a right-side region of a wearer of the garment and to thereby detect muscle activity of one or more muscles in the right-side region. The second electrodes may be left-side electrodes arranged to contact the skin of a left-side region of a wearer of the garment and to thereby detect muscle activity of one or more muscles in the left-side region. For example, the first (e.g. right-side) electrodes may be arranged to contact the skin of the wearer's right limb, e.g. right leg or right arm and the second (e.g. left-side) electrodes may be arranged to contact the skin of the wearer's left limb, e.g. left leg or left arm. It may be that the first (e.g. right-side) electrodes may be arranged to contact the skin of the wearer at least partially over a muscle in the first region (for example, a right hamstring or a right bicep). It may be that the second (e.g. left-side) electrodes are arranged to contact the skin of the wearer at least partially over a muscle in the second region, optionally corresponding to an equivalent muscle in the first region (for example, a left hamstring, where the first electrodes are arranged to contact the skin of the wearer at least partially over the right hamstring, or a left bicep, where the first electrodes are arranged to contact the skin of the wearer at least partially over the right bicep).

However, in some examples, the first electrodes may be arranged to contact the skin of the wearer to thereby detect muscle activity in a first region and the second electrodes may be arranged to detect muscle activity in a second region, where both the muscles in the first region and the muscles in the second region are expected to be activated during an exercise. For example, it may be that the first electrodes are arranged to contact the skin of the wearer to thereby detect muscle activity in a first region and the second electrodes are arranged to detect muscle activity in a second region, where both the muscles in the first region and the muscles in the second region are complimentary muscles. It will be understood that complimentary muscles are muscles which are used (e.g. together) when performing a particular exercise.

It may be the case that the muscles in the first region are right-side muscles and the muscles in the second region are (optionally corresponding, equivalent, or complimentary) left-side muscles, however this is not required. For example, if the wearer is performing a plank and their abdominal muscles are weaker or fatigued, they may (e.g. unintentionally) engage muscles in their back in order to make the plank easier to perform. Accordingly, in this example, the first region may be a region of the wearer's abdomen (and optionally the detected muscle activity may be activity of one or more abdominal muscles) and the second region may be a region of the wearer's dorsum (and optionally the detected muscle activity may be activity of one or more muscles in the wearer's back). In a further example, the first or second region may be a region of the wearer's chest.

The controller may be configured to receive a first (e.g. right-side) activity signal indicative of detected activity of one or more muscles in the first (e.g. right-side) region from the first (e.g. right-side) electrodes. The controller may be configured to receive a second (e.g. left-side) activity signal indicative of detected activity of one or more muscles in the second (e.g. left-side) region from the second (e.g. left-side) electrodes. The controller may be configured to determine one or more indicators of relative performance of the one or more muscles in the first (e.g. right-side) region and the one or more muscles in the second (e.g. left-side) region, in dependence on the first (e.g. right-side) activity signal and the second (e.g. left-side) activity signal.

The controller may be configured to output the one or more indicators of relative performance.

The method may comprise receiving a first (e.g. right-side) activity signal indicative of detected activity of one or more muscles in the first (e.g. right-side) region from the first (e.g. right-side) electrodes. The method may comprise receiving a second (e.g. left-side) activity signal indicative of detected activity of one or more muscles in the second (e.g. left-side) region from the second (e.g. left-side) electrodes. The method may comprise determining one or more indicators of relative performance of the one or more muscles in the first (e.g. right-side) region and the one or more muscles in the second (e.g. left-side) region in dependence on the first (e.g. right-side) activity signal and the second (e.g. left-side) activity signal. The method may comprise outputting the one or more indicators of relative performance.

The determination of one or more indicators of relative performance of muscles as described hereinbefore is itself believed to be novel and so, in a further aspect of the invention, there is provided apparatus comprising a garment, the garment comprising:
at least two first (e.g. right-side) electrodes arranged to contact the skin of a first (e.g. right-side) region of a wearer of the garment and to thereby detect muscle activity of one or more muscles in the first (e.g. right-side) region;
at least two second (e.g. left-side) electrodes arranged to contact the skin of a second (e.g. left-side) region of a wearer of the garment and to thereby detect muscle activity of one or more muscles in the second (e.g. left-side) region;
the apparatus further comprising a controller configured to:
receive a first (e.g. right-side) activity signal indicative of detected muscle activity of the one or muscles in the first (e.g. right-side) region from the first (e.g. right-side) electrodes;
receive a second (e.g. left-side) activity signal indicative of detected muscle activity of the one or more muscles in the second (e.g. left-side) region from the second (e.g. left-side) electrodes;
determine one or more indicators of relative performance of the one or more muscles in the first (e.g. right-side) region and the one or more muscles in the second (e.g. left-side) region, in dependence on the first (e.g. right-side) activity signal and the second (e.g. left-side) activity signal. The controller may be configured to output the one or more indicators of relative performance.

The further aspect of the present invention extends to a method of use of apparatus comprising a garment, wherein the garment is worn by a wearer. The garment comprises: at least two first (e.g. right-side) electrodes arranged to contact the skin of a first (e.g. right-side) region of a wearer of the garment and to thereby detect muscle activity of one or more muscles in the first (e.g. right-side) region; and at least two second (e.g. left-side) electrodes arranged to contact the skin of a second (e.g. left-side) region of a wearer of the garment and to thereby detect muscle activity of one or more muscles in the second (e.g. left-side) region. The method comprises receiving a first (e.g. right-side) activity signal indicative of detected activity of one or more muscles in the first (e.g. right-side) region from the first (e.g. right-side) electrodes; receiving a second (e.g. left-side) activity signal indicative of detected activity of one or more muscles in the second (e.g. left-side) region from the second (e.g. left-side) electrodes; determining one or more indicators of relative performance of the one or more muscles in the first (e.g. right-side) region and the one or more muscles in the second (e.g. left-side) region in dependence on the first (e.g. right-side) activity signal and the second (e.g. left-side) activity signal. The method may comprise outputting the one or more indicators of relative performance.

Providing corresponding first (e.g. right-side) and second (e.g. left-side) electrodes allows a comparison between the activation of the muscles in a first (e.g. right-side) region and the muscles in a second (e.g. left-side) region of the wearer's body. This can be particularly helpful where a wearer is recovering from an injury, as it allows an assessment of how effectively an injured muscle or muscle group is being activated during an exercise, in comparison to a corresponding uninjured muscle. This can also be used to assess whether a wearer is activating muscles which should not be activated during an exercise (e.g. as described above, where a wearer might engage muscles in their back whilst performing a plank).

The right-side region of the wearer may be a right limb. The right-side region of the wearer may be a right arm. The right-side region may be a right shoulder. The right-side region of the wearer may be a right leg. The right-side region of the wearer may be a right hip. The right-side region of the wearer may be a right buttock. The right-side region of the wearer may be a muscle (optionally a group of muscles) of the right-hand side of the wearer's body. The right-side region may comprise one or more muscles in the right-hand side of the wearer's abdomen. The right-side region may comprise one or more muscles in the right-hand side of the wearer's chest. The right-side region may comprise one or more muscles in the right-hand side of the wearer's back. The left-side region of the wearer may be a left limb. The left-side region of the wearer may be a left arm. The left-side region of the wearer may be a left leg. The left-side region of the wearer may be a left hip. The left-side region of the wearer may be a left buttock. The left-side region of the wearer may be a muscle (optionally a group of muscles) of the left-hand side of the wearer's body. The left-side region may comprise one or more muscles in the left-hand side of the wearer's abdomen. The left-side region may comprise one or more muscles in the left-hand side of the wearer's chest. The left-side region may comprise one or more muscles in the left-hand side of the wearer's back.

The garment may comprise at least six first (e.g. right-side) electrodes. The garment may comprise at least six second (e.g. left-side) electrodes. At least one first (e.g. right-side) electrode may form an anode-cathode pair with at least one other first (e.g. right-side) electrode. At least one second (e.g. left-side) electrode may form an anode-cathode pair with at least one other second (e.g. left-side) electrode. It may be that each first (e.g. right-side) electrode may form an anode-cathode pair with another first (e.g. right-side) electrode. It may be that each second (e.g. left-side) electrode may form an anode-cathode pair with another second (e.g. left-side) electrode.

In an example, the garment may comprise at least two first electrodes arranged to be positioned at least partially over a first quadricep in a first leg. The garment may (e.g. further) comprise at least two first electrodes arranged to be positioned at least partially over a second quadricep in the first leg. The garment may comprise at least two (e.g. further) first electrodes arranged to be positioned at least partially over a hamstring in the first leg. The garment may comprise at least two second electrodes arranged to be positioned at least partially over a first quadricep in a second leg. The garment may comprise at least two (e.g. further) second electrodes arranged to be positioned at least partially over a second quadricep in the second leg. The garment may comprise at least two (e.g. further) second electrodes arranged to be positioned at least partially over a hamstring in the second leg.

In a further example, the garment may comprise an array of electrodes comprising a plurality of electrodes (optionally arranged in a grid) wherein each electrode in the array of electrodes is arranged such that it may (optionally temporarily) form an anode-cathode pair with any other electrode in the array of electrodes. Advantageously this allows many options for detecting activity of substantially any muscle at least partially covered by the garment.

The array of electrodes may comprise sufficient electrodes that more than two (e.g. three or more, optionally four or more) electrodes are positioned at least partially over one muscle (e.g. the vastus lateralis). Advantageously, this allows for receiving two or more activity signals indicating the activity of the said one muscle. Accordingly, the controller may be configured to determine (or the method may comprise determining) the strongest (e.g. highest amplitude) signal of the two or more activity signals indicating the activity of the said one muscle. The controller may be configured to determine (or the method may comprise determining) the cleanest signal (e.g. the signal having the highest signal to noise ratio) of the two or more activity signals 4 indicating the activity of the said one muscle. The controller may be configured to cause (or the method may comprise causing) the electrical visual indicator device to output a visual indication of the detective activity of the muscle in dependence on the strongest signal. The controller may be configured to cause (or the method may comprise causing) the electrical visual indicator device to output a visual indication of the detective activity of the muscle in dependence on the cleanest signal.

The garment may comprise one or more locating structures. The garment may comprise a plurality of locating structures, each associated with an electrode. For example, the garment may comprise as many locating structures as there are electrodes. The garment may comprise a locating structure for each electrode. The or each locating structure is typically configured to increase the frictional forces between the garment and the skin of a wearer of the garment to thereby limit the movement of the electrodes (e.g. each respective electrode) relative to the skin of the wearer. For example, the or each locating structure may comprise an outermost surface configured to increase the frictional forces between the garment and the skin of the wearer of the garment to thereby limit the movement of the electrodes (e.g. each respective electrode) relative to the skin of the wearer (e.g. during exercise)

The limitation of movement of electrodes in a garment hereinbefore described is itself believed to be novel and so, in a further aspect of the invention, there is provided apparatus comprising a garment, the garment comprising:
one or more electrodes; and
at least one locating structure (e.g. one locating structure per electrode), the locating structure comprising an outermost surface configured to increase the frictional forces between the garment and the skin of a wearer of the garment to thereby limit the movement of the electrodes (e.g. the respective electrode) relative to the skin of the wearer.

The muscle activity sensors detect muscle activity more accurately if the electrodes are properly positioned and if the extent to which they can move, relative to the wearer's skin, during exercise is limited. The provision of locating structures which increase the frictional forces between the garment and the wearer's skin limits the extent to which the electrodes can move relative to the wearer's skin. Accordingly, by providing a garment which includes such locating structures improves accuracy of muscle activity detection.

The locating structure may be defined on an inner surface of the garment, the inner surface configured to be in contact with the skin of the wearer when the garment is worn by the wearer.

The locating structure may comprise an elastomer material. The locating structure may comprise a solid elastomer material. The locating structure may comprise silicone. The locating structure may comprise a silicone patch. The locating structure may comprise rubber (e.g. latex). The locating structure may comprise a rubber (e.g. latex) patch.

Elastomer materials such as silicone and latex are relatively flexible and provide grip. As such, a locating structure comprising an elastomer material is more comfortable than a relatively more rigid locating structure, whilst helping to limit movement of the electrodes relative to the skin of the wearer.

The garment may be (e.g. at least partially) fabricated from a first textile having a first elasticity (e.g. a first elastic modulus). The garment may comprise a further layer, such as a compression layer, (e.g. at least partially) covering the first textile. The further layer (e.g. the compression layer) may have a second elasticity (e.g. a second elastic modulus) different to (e.g. greater than) the first elasticity. The further layer may define one or more of apertures therein. The further layer may cover at least 5% of the garment, optionally at least 20%, optionally at least 30% (i.e. excluding the apertures). The further layer may define a grid structure, optionally a hexagonal grid structure. The compression layer may comprise one or more resiliently deformable band portions, each resiliently deformable band portion configured to apply elastic resistance to the skin wearer to thereby limit the movement of one or more electrodes relative to the skin of the wearer. The or each resiliently deformable band portion also limits the extent to which electrodes move relative to the wearer's skin during exercise. The or each resiliently deformable band portion may be a non-continuous resiliently deformable band portion.

The controller may be configured to receive an estimate of the centre of gravity of the wearer. The method may comprise receiving an estimate of the centre of gravity of the wearer. It is helpful to know the centre of gravity of a wearer when assessing the performance of exercises. It will be understood that the estimate of the centre of gravity may be an estimate of a point (e.g. a point in three-dimensional space). However, the estimate of the centre of gravity may be an estimate of a line (e.g. a line extending through 2-dimensional space). For example, the estimate of the centre of gravity may be a (e.g. vertical) line (or optionally a vertical plane) extending through the expected location of the wearer of the garment (e.g. through the three-dimensional centre of gravity of the wearer of the garment), from a region of the wearer's feet to a region of the wearer's head. Alternatively the estimate of the centre of gravity may be a (e.g. horizontal) line (or optionally a lateral plane) extending laterally through the expected location of the wearer of the garment (e.g. through the three-dimensional centre of gravity of the wearer of the garment), in a direction parallel to a line passing from a region of the wearer's left hip to a region of the wearer's right hip. Alternatively the estimate of the centre of gravity may be a (e.g. horizontal) line extending laterally through the expected location of the wearer of the garment (e.g. through the three-dimensional centre of gravity of the wearer of the garment), in a direction parallel to a line passing from a region of the wearer's abdomen to a region of the wearer's back at substantially the same height as the region of the wearer's abdomen. In some embodiments, in may be that the estimate of the centre of gravity is an estimate of the centre of gravity within a three-dimensional space. In the case of a line, or a plane, it will be understood that the line defines two of the three-dimensional constraints necessary to define the three-dimensional centre of gravity, and the plane defines one of the three-dimensional constraints necessary to define the three-dimensional centre of gravity. Nevertheless, even one or two of the dimensional constraints necessary to define the three-dimensional centre of gravity can still be considered to be an estimate of a centre of gravity.

The computer readable memory (e.g. the biometric data store) may comprise biometric data (e.g. biometric information about the wearer of the garment). The biometric data (e.g. information) may comprise an estimate of the total weight of the wearer. The biometric data (e.g. information) may comprise an estimate of the combined weight of the right limbs of the wearer. The biometric data (e.g. information) may comprise an estimate of the combined weight of the left limbs of the wearer. The biometric data (e.g. information) may comprise an estimate of the width of the feet of the wearer. The biometric data (e.g. information) may comprise a predetermined separation distance.

The biometric data (e.g. information) may comprise an expected total weight of a human (optionally a human of the same age and/or gender as the wearer). The biometric data (e.g. information) may comprise an expected combined weight of the right limbs of a human (optionally a human of the same age and/or gender and/or weight as the wearer). The biometric data (e.g. information) may comprise an expected combined weight of the left limbs of a human (optionally a human of the same age and/or gender and/or weight as the wearer). The biometric data (e.g. information) may comprise an expected width of the feet of a human (optionally a human of the same age and/or gender as the wearer).

The computer readable memory (e.g. the biometric data store) may comprise one or more data structures together indicative of an estimate of the total weight of the wearer. The computer readable memory (e.g. the biometric data store) may comprise one or more data structures together indicative of a body fat indicator indicative of a proportion of body fat of the wearer (e.g. as a proportion of a total weight of the wearer or in relation to the proportion of the wearer not made up of body fat). The computer readable memory (e.g. the biometric data store) may comprise one or more data structures together indicative of an estimate of the body fat percentage of the wearer.

The computer readable memory (e.g. the biometric data store) may comprise one or more data structures together indicative of an estimate of a width of the feet of the wearer. For example, a width of the feet of the wearer may be an estimate of the average (e.g. mean) width of the feet across the widest part of the sole of each foot of the wearer (e.g. a width generally orthogonal to a length as measured from a heel region to a toe region). A width of the feet of the wearer may be an estimate of the average (e.g. mean) width of the feet where the toes meet the sole of each foot of the wearer.

The computer readable memory (e.g. the biometric data store) may comprise one or more data structures together indicative of a predetermined separation distance. The predetermined separation distance may be a known distance by which the wearer would space their feet when told to stand with their feet shoulder-width apart. For example, the predetermined separation distance may be a distance between the outermost lateral part of the right foot of the wearer and the outermost lateral part of the left foot of the wearer (e.g. when the wearer spaces their feet shoulder-width apart). In an example, the separation distance may be at least 20 centimetres, e.g. at least 30 centimetres, e.g. at least 40 centimetres. The separation distance may be no more than 80 centimetres, e.g. no more than 70 centimetres, e.g. no more than 60 centimetres. The skilled person will appreciate that whilst the separation distance may vary in dependence on the anatomy of the wearer, this variation is within the scope of the invention.

The controller may be configured receive one or more of the one or more data structures from the computer readable memory (e.g. the biometric data store). The controller may be configured to determine an estimate of the centre of gravity of the wearer in dependence on the one or more received data structures. The controller may be configured to output the estimate of the centre of gravity. The controller may be configured to output the estimate of the centre of gravity to the biometric data store.

The method may comprise receiving one or more of the one or more data structures from the computer readable memory (e.g. the biometric data store). The method may comprise determining an estimate of the centre of gravity of the wearer in dependence on the one or more received data structures. The method may comprise outputting the estimate the centre of gravity. For example, the method may comprise outputting the estimate of the centre of gravity to the computer readable memory (e.g. the biometric data store).

In an example, the centre of gravity, C_{G}, may be determined as follows:

$${C}_{G} = \frac{{M}_{4}}{{W}_{4}} .$$

Where M₄ is the total moment and W₄ is the total weight of the wearer of the garment. For example:
   W₁ is a first weight and may be the combined weight of the right arm and the right leg of the wearer (this may be determined, for example, on the basis of the estimate of the total weight of the wearer, the body fat indicator or the estimate of the body fat percentage of the wearer, and information about typical proportions of adult humans);
   W₂ is a second weight and may be the combined weight of the left arm and the left leg of the wearer;
   W₃ is a third weight and may be the weight of the wearer excluding the weight of the right leg, the right arm, the left leg, and the left arm;
   W₄ is a fourth weight and may be the total weight of the wearer;
   D₁ is a first distance and may be the separation distance divided by 2;
   D₂ is a second distance and may be the width of the wearer's foot divided by 2;
   D₃ is a third distance and may be the separation distance minus the width of the wearer's foot divided by 2;
   M₁ is a first moment and may be found as follows: M₁=W₁×D₁;
M₂ is a second moment and may be found as follows: M₂=W₂×D₂;
M₃ is a third moment and may be found as follows: M₃=W₃×D₃;
M₄ is the total moment and may be found as follows: M₄=M₁+M₂+M₃.

The skilled person will appreciate that the above example is provided for illustrative purposes only, and that there are many ways in which the centre of gravity of a wearer might be determined.

The determination of the centre of gravity of a wearer as hereinbefore described is itself believed to be novel and so, in a further aspect of the invention, there is provided apparatus comprising a garment,
the apparatus further comprising a computer readable memory (e.g. a non-transitory computer readable storage medium) comprising a biometric data store (e.g. database), the biometric data store comprising one or more data structures together indicative of:
   an estimate of the total weight of the wearer;
   a predetermined separation distance,
the apparatus further comprising a controller, the controller configured to:
   receive one or more of the one of more data structures from the biometric data store; and
   determine an estimate of the centre of gravity of the wearer in dependence on the one or more received data structures. The controller may be configured to output the estimate of centre of gravity. The one or more data structures may (e.g. further) be together indicative of a body fat indicator indicative of an amount (e.g. a proportion) of body fat of the wearer (e.g. as a proportion of a total weight of the wearer or in relation to the proportion of the wearer not made up of body fat). The one or more data structures may (e.g. further) be together indicative of an estimate of the body fat percentage of the wearer. The one or more data structures may (e.g. further) be together indicative of an estimate of the width of the feet of the wearer. The one or more data structures may (e.g. further) be together indicative of an estimate of the height of the wearer.

The further aspect of the present invention extends to a method of use of apparatus comprising a garment, wherein the garment is worn by a wearer. The apparatus comprises a computer readable memory (e.g. a non-transitory computer readable storage medium). The computer readable memory may comprise a biometric data store (e.g. database). The computer readable memory (e.g. the biometric database) comprises one or more data structures together indicative of:
an estimate of the total weight of the wearer;
a body fat indicator indicative of an amount (e.g. a proportion) of body fat of the wearer (e.g. as a proportion of a total weight of the wearer or in relation to the proportion of the wearer not made up of body fat), optionally an estimate of the body fat percentage of the wearer;
an estimate of a width of the feet of the wearer; and
a predetermined separation distance.

The method comprises receiving one or more of the one or more data structures from the computer readable memory (e.g. the biometric data store). The method comprises determining an estimate of the centre of gravity of the wearer in dependence on the one or more received data structures. The method may comprise outputting the estimate the centre of gravity. For example, the method may comprise outputting the estimate of the centre of gravity to the computer readable memory (e.g. the biometric data store).

The method may comprise determining the predetermined separation distance. For example, the method may comprise the wearer positioning their feet, determining the separation distance between their feet, and outputting the determined separation distance (e.g. to the biometric data store).

A garment which can be used to estimate the centre of gravity of the wearer is helpful in part because centre of gravity determination (or estimation) can be complex for a person to calculate. Furthermore, once an estimate of the centre of gravity of the wearer is known, this information can be used to monitor their movements relative to their centre of gravity during exercise. This is helpful in determining whether exercises are being performed as intended.

The apparatus (optionally the garment) may comprise at least one motion sensor for detecting motion of the wearer (e.g. when performing an exercise). The at least one motion sensor may comprise at least one accelerometer. The at least one motion sensor may comprise at least one gyroscope. The at least one motion sensor may comprise a magnetometer. The at least one motion sensor may comprise a compass. The at least one motion sensor may comprise a tilt sensor. The apparatus (optionally the garment) may comprise an inertial measurement unit (IMU). The apparatus (optionally the garment) may comprise at least two motion sensors, optionally exactly two motion sensors.

In some embodiments, the garment may be a pair of shorts. In some embodiments, the shorts may comprise the motion sensor (optionally accelerometer, gyroscope, magnetometer, compass, tilt sensor, IMU). The motion sensor may be mounted on or integrated into the rear of the shorts, optionally in an uppermost region of the shorts, for example close to and below the waistband. The motion sensor may be mounted on or integrated into the rear of the shorts and the shorts may be configured so that the motion sensor is close to (e.g. within 8 cm of, e.g. within 5 cm, e.g. within 3 cm) the medial part of the wearer's lower back, when the shorts are worn. In some embodiments, the garment may comprise a plurality of motion sensors, for example one motion sensor mounted on or integrated with each leg, or for each sleeve, and optionally other motion sensors mounted on or integrated with other regions of the garment.

The controller may be configured to receive a motion signal indicative of motion of the wearer from the at least one motion sensor. The motion signal may comprise an accelerometer signal. The motion signal may comprise a gyroscope signal. The motion signal may comprise a magnetometer signal. The motion signal may comprise a compass signal. The motion signal may comprise an IMU signal. The controller may be configured to determine a distance from the centre of gravity that the wearer moves when performing an exercise, in dependence on the motion signal. The controller may be configured to output the determined distance from the centre of gravity. The controller may be configured to determine the direction in which the wearer moves when performing the exercise, in dependence on the motion signal. The controller may be configured to output the determined direction.

The method may comprise receiving a motion signal indicative of motion of the wearer from the at least one motion sensor. The method may comprise determining a distance from the estimated centre of gravity that the wearer moves when performing an exercise, in dependence on motion signal. The method may comprise outputting the determined distance from the centre of gravity. The method may comprise determining the direction in which the wearer moves when performing the exercise, in dependence on the motion signal. The method may comprise outputting the determined direction.

Apparatus which can be used in this way to monitor the movements of a wearer relative to their centre of gravity during exercise is helpful in determining whether exercises are being performed as intended.

The apparatus (e.g. the controller) may be configured to prompt (and the method may comprise prompting) a user (e.g. wearer) to perform one or more motion sensor calibration steps when the shorts are donned for the first time in a day, e.g. before any exercises are performed. The apparatus may be configured to prompt (and the method may comprise prompting) a user (e.g. wearer) to perform one or more motion sensor calibration steps in between performing exercises. The motion sensor calibration steps may comprise one or more of:
confirming the location of the motion sensor (e.g. the IMU) relative to or on the wearer;
confirming that the wearer is in a standing position meeting predetermined criteria (e.g. criteria which may include one or more of: the wearer is standing with their feet at a specific separation distance, e.g. hip-width; the wearer is in a relaxed position; the wearer's feet are both in contact with the ground at both the ball of the foot and the heel of the foot; the wearer is facing directly forward and not twisting their torso or turning their head; the wearer's arms are by their sides, etc);
the wearer maintaining the standing position for at least 1 second (e.g. at least 2 seconds, e.g. at least 5 seconds, typically less than 10 seconds).

These calibration steps may be used to estimate a starting orientation (e.g. of the wearer, optionally of the motion sensor). The calibration steps may be used to estimate a starting position (e.g. of the wearer, optionally of the motion sensor). It will be understood that the starting orientation and/or starting position of the motion sensor may be used to estimate the starting orientation and/or starting position of the wearer, when the wearer is wearing the garment and the motion sensor is mounted on (or integrated with) the garment. The apparatus (e.g. the controller) may be configured to receive a motion signal indicative of motion of the wearer from the at least on motion sensor. The motion signal may comprise an accelerometer signal. The motion signal may comprise a gyroscope signal. The motion signal may comprise a magnetometer signal. The motion signal may comprise a compass signal. The motion signal may comprise an IMU signal.

The apparatus (e.g. the controller) may be configured to determine (and the method may comprise determining) that the motion sensor has moved from an intended location, in dependence on the motion signal. For example, it may be that the motion signal is indicative of expected movements, but with an offset, and this offset may be due to the motion sensor having moved. Where it is determined that the motion sensor has moved from an intended location, the apparatus (e.g. the controller) may be configured to prompt (and the method may comprise prompting) the user to reposition the motion sensor (e.g. by adjusting the garment) and optionally to repeat the motion sensor calibration steps. In an example, the intended location may be a location close to (e.g. within 8 cm of, e.g. within 5 cm, e.g. within 3 cm) the medial part of the wearer's lower back, e.g. when the garment is a pair of shorts and the sensor is mounted to or integrated with an upper, rear, central part of the shorts.

The apparatus (e.g. the controller) may be configured to (and method may comprise) estimate a starting orientation (optionally a starting position), e.g. in dependence on the motion signal when the wearer is at rest, for example in dependence on the motion sensor calibration steps. The apparatus (e.g. the controller) may be configured to estimate a change in orientation (and/or a distance) from the starting orientation (and/or starting position), of the wearer (optionally of the motion sensor) in dependence on the motion signal when the wearer performs an exercise. The controller may be configured to output the estimated change in orientation (and/or distance) from the starting orientation (and/or starting position). The controller may be configured to estimate the (e.g. range of) angles through which the wearer moves (e.g. relative to the starting orientation) when performing the exercise, in dependence on the motion signal. The controller may be configured to estimate the distance and/or direction in which the wearer moves (e.g. relative to the starting orientation and/or starting position) when performing the exercise, in dependence on the motion signal. The controller may be configured to output the estimated (e.g. range of) angles and/or the estimated distance and/or direction.

The estimated (e.g. range of) angles may comprise angles of rotation around an x-axis (e.g. with an angle of 0° defined at the starting orientation, up to ±180°). The estimated (e.g. range of) angles may comprise angles of rotation around a y-axis (e.g. with an angle of 0° defined at the starting orientation up to ±180°). The estimated (e.g. range of) angles may comprise angles of rotation around a z-axis (e.g. with an angle of 0° defined at the starting orientation up to ±180°). In other words, the estimated (e.g. range of) angles may comprise Euler angles. The estimated (e.g. range of) angles may comprise pitch and/or roll and/or yaw angles.

The method may comprise receiving a motion signal indicative of motion of the wearer from the at least one motion sensor. The method may comprise estimating a starting orientation (and/or a starting position) in dependence on the motion signal when the wearer is at rest, for example in dependence on the motion signal calibration steps. The method may comprise estimating a change in orientation (and/or a distance) from the starting orientation (and/or starting position), of the wearer (optionally of the motion sensor) in dependence on the motion signal, when the wearer performs an exercise. The method may comprise outputting the change in orientation (and/or distance) from the starting orientation (and/or starting position). The method may comprise estimating the (e.g. range of angles) through which the wearer moves (e.g. relative to the starting orientation) when performing the exercise, in dependence on the motion signal. The method may comprise estimating the direction in which the wearer moves (e.g. relative to the starting orientation and/or starting position) when performing the exercise, in dependence on the motion signal. The method may comprise outputting the estimated (e.g. range of) angles and/or the estimated direction.

Apparatus which can be used in this way to monitor the movements of a wearer relative to the starting orientation and/or starting position during exercise is helpful in determining whether exercises are being performed as intended.

The controller may be configured to determine when a movement of the wearer is below a predetermined movement threshold in dependence on the motion signal. The method may comprise determining when a movement of the wearer is below a predetermined movement threshold in dependence on the motion signal. If the movement of the wearer is below a predetermined movement threshold, this may be indicative that the wearer is substantially stationary (e.g. standing still).

The controller may be configured to determine (and the method may comprise determining) a spatial reference. The spatial reference may comprise (e.g. be) the estimated centre of gravity. The spatial reference may comprise (e.g. be) the estimated starting position. The spatial reference may comprise (e.g. be) the estimated starting orientation. The spatial reference may comprise (e.g. be) a location, for example as defined by a set of coordinates in space. While the spatial reference may comprise a reference point, the skilled person will appreciate that it need not be a single point in space and may comprise a region, for example. The spatial reference may comprise (e.g. be) an orientation, for example as defined by a set of angles around x-, y-, and z-coordinates. The spatial reference may comprise a location and an orientation.

The controller may be configured to output an alert if the wearer moves more than a predetermined distance from the spatial reference (e.g. in a particular direction or through a particular range of angles). The method may comprise outputting an alert if the wearer moves more than a predetermined distance from the spatial reference (e.g. in a particular direction or through a particular range of angles).

In an example, the estimate of the centre of gravity may be a vertical plane extending through the expected location of the wearer of the garment from a region of the wearer's feet to a region of the wearer's head (e.g. parallel to the height of the wearer when standing and perpendicular to a line between the wearer's right shoulder and the wearer's left shoulder). The controller may be configured to determine (and the method may comprise determining) when the wearer moves more than a predetermined distance from the said vertical plane. The controller may be configured to output (and the method may comprise outputting) an alert when the wearer moves more than a predetermined distance from the said vertical plane.

The alert may comprise a visual alert. For example, outputting an alert may comprise causing the at least one electrical visual indicator device to output an alert visual indication (e.g. the or each electrical visual indicator is caused to illuminate (e.g. output light) in a red colour). The alert may comprise an alert displayed on a mobile device, such as a laptop, tablet, smartphone, or smart watch. The alert may comprise an audio alert. For example, outputting an alert may comprise sounding an alarm. The alert may comprise a haptic alert. For example, the apparatus (optionally the garment) may comprise a haptic feedback device configured to provide haptic feedback to the wearer, and outputting an alert may comprise causing the haptic feedback device to output haptic feedback.

By providing an alert when a wearer moves more than a predetermined distance from the spatial reference, it is possible to indicate to the wearer that they are not performing their exercise correctly.

The controller may be configured to cause (or the method may comprise causing) a first light emitter (e.g. LED) to illuminate if the wearer is moving within a first range of the spatial reference and causing a second light emitter (e.g. LED) to illuminate (and optionally causing the first light emitter to stop illuminating) if the wearer moves within a second range of the spatial reference where the first range is closer to the spatial reference than the second range. Where a plurality of light emitters are arranged in a pattern (e.g. in a series of lines), the controller may be configured to cause (or the method may comprise causing):
one or more first light emitters (e.g. LEDs) to illuminate if the wearer is moving within a first range of the spatial reference;
one or more second light emitters (e.g. LEDs) to illuminate if the wearer moves within a second range of the spatial reference where the first range is closer to the spatial reference than the second range (and optionally causing the as the first light emitter(s) to stops illuminating);
and optionally causing one or more third light emitters (e.g. LEDs) to illuminate if the wearer moves within a third range of the spatial reference where the first and second ranges are both closer to the spatial reference than the third range (and optionally causing the as the first light emitter(s) to stops illuminating).

The second light emitter(s) may be (e.g. laterally) between the first and third light emitter(s). In this way a “flowing effect” can be achieved, with light emitters illuminating in sequence. For example, where the light emitters are arranged in a series of lines, the first light emitters may be arranged in one or more medial lines (e.g. lines closest to the centre of the wearer), the third light emitters may be arranged in one or more lateral lines (e.g. lines furthest from the centre of the wearer), and the second light emitters may be arranged in one or more lines laterally between the first and third light emitters.

The controller may be configured to determine when an exercise has been carried out in dependence on the motion signal. The controller may be configured to determine the frequency with which the exercise is carried out in a period and optionally thereby determine an estimate of the rate at which the exercise is carried out. The controller may be configured to output the estimated rate at which the exercise is carried out.

The method may comprise determining when an exercise has been carried out in dependence on the motion signal. The method may comprise determining the frequency with which the exercise is carried out in a period and optionally thereby determining an estimate of the rate at which the exercise is carried out. The method may comprise outputting the estimated rate at which the exercise is carried out.

The exercise data may comprise a predetermined optimal rate at which an exercise should be carried out (e.g. by the wearer). The controller may be configured to compare the estimate of the rate at which the exercise is carried out and the predetermined optimal rate at which the exercise should be carried out. The controller may be configured to output an alert if the wearer is performing the exercise at a rate that is higher or lower than the predetermined optimal rate. For example, the controller may be configured to output an alert if the wearer is performing the exercise at a rate more than 10% of the predetermined optimal rate, e.g. more than 15% of the predetermined optimal rate, e.g. more than 30% of the predetermined optimal rate. The controller may be configured to output an alert if the wearer is performing the exercise at a rate less than 30% of the predetermined optimal rate, e.g. less than 15% of the predetermined optimal rate, e.g. less than 10% of the predetermined optimal rate.

The method may comprise comparing the estimate of the rate at which the exercise is carried out and the predetermined optimal rate at which the exercise should be carried out. The method may comprise outputting an alert if the wearer is performing the exercise at a rate that is higher or lower than the predetermined optimal rate. For example, the method may comprise outputting an alert if the wearer is performing the exercise at a rate more than 10% of the predetermined optimal rate, e.g. more than 15% of the predetermined optimal rate, e.g. more than 30% of the predetermined optimal rate. The method may comprise outputting an alert if the wearer is performing the exercise at a rate less than 30% of the predetermined optimal rate, e.g. less than 15% of the predetermined optimal rate, e.g. less than 10% of the predetermined optimal rate

The controller may be configured to determine a first duration between a first repetition of the exercise and a second repetition of the exercise. The controller may be configured to determine a second duration between the second repetition of the exercise and a third repetition of the exercise. The controller may be configured to compare the first duration and the second duration. The controller may be configured to output an alert if the first duration has a length of less than 90% of the second duration, e.g. less than 80% of the second duration, e.g. less than 70% of the second duration. The controller may be configured to output an alert if the first duration has a length of 10% more than the second duration, e.g. 20% more than the second duration, e.g. 30% more than the second duration.

The method may comprise determining a first duration between a first repetition of the exercise and a second repetition of the exercise. The method may comprise determining a second duration between the second repetition of the exercise and a third repetition of the exercise. The method may comprise comparing the first duration and the second duration. The method may comprise outputting an alert if the first duration has a length of less than 90% of the second duration, e.g. less than 80% of the second duration, e.g. less than 70% of the second duration. The method may comprise outputting an alert if the first duration has a length of 10% more than the second duration, e.g. 20% more than the second duration, e.g. 30% more than the second duration.

It is generally beneficial if repetitions of exercises are performed at a consistent rate, with consistent periods between repetitions. An apparatus, and particularly a garment, that can be used indicate to the wearer if their repetitions of an exercise are not consistent, thus helps the user to obtain improved health benefits from their exercises.

The controller may be configured to receive (or the method may comprise receiving) an activity signal indicative of detected activity of a (e.g. the) muscle from the at least one muscle activity sensor. The controller may be configured to receive (or the method may comprise receiving) a motion signal (optionally an accelerometer signal) indicative of motion of the wearer from the at least one motion sensor. The controller may be configured to determine (or the method may comprise determining) a degree of muscle fatigue in dependence on the activity signal. The controller may be configured to determine (or the method may comprise determining) an estimate of a parameter indicative of the degree of muscle fatigue (e.g. further) in dependence on the motion signal (optionally an accelerometer signal). The controller may be configured to output (or the method may comprise outputting) the estimate of the parameter indicative of the degree of muscle fatigue.

In an example, the controller may cause (or the method may comprise causing) the at least one electrical visual indicator device to output a visual indication of the estimate of the parameter indicative of the degree of muscle fatigue. The controller may cause (or the method may comprise causing) the at least one electrical visual indicator device to illuminate (e.g. steadily, without flashing or repeatedly switching on and off) if the estimate of the parameter indicative of the degree of muscle fatigue is indicative of the muscle not being fatigued. The controller may cause (or the method may comprise causing) the at least one electrical visual indicator device to flash at a first frequency (e.g. to illuminate, and then to cease illuminating, and then to illuminate, repeatedly) if the estimate of the parameter indicative of the degree of muscle fatigue is indicative of a first degree of muscle fatigue, and to flash at a second frequency, higher than the first frequency if the estimate of the parameter indicative of the degree of muscle fatigue is indicative of a second degree of muscle fatigue which is a greater degree of muscle fatigue than the first degree of muscle fatigue. The controller may cause (or the method may comprise causing) the at least one electrical visual indicator device to flash at one or more further frequencies if the estimate of the parameter indicative of the degree of muscle fatigue is indicative of a one or more further degrees of muscle fatigue.

In an example, where the controller causes (or the method comprises causing) the at least one electrical visual indicator device to flash at a first frequency, this may comprise causing the at least one electrical visual indicator device to illuminate for at least 3 seconds (e.g. at least 4 seconds, optionally at least 5 seconds), and then causing the at least one electrical visual indicator device to stop illuminating for at least 3 seconds (e.g. at least 4 seconds, e.g. at least 5 seconds). Where the controller causes (or the method comprising causing) the at least one electrical visual indicator device to flash at a second frequency, this may comprise causing the at least one electrical visual indicator device to illuminate for at least 1 second (e.g. at least 2 seconds, optionally at least 3 seconds), and then causing the at least one electrical visual indicator device to stop illuminating for at least 0.5 seconds (e.g. at least 1 second, e.g. at least 2 seconds). However, the skilled person will appreciate that other options may also be suitable. Furthermore, it will be understood that other ways of visual indicating an estimate of the parameter indicative of the degree of muscle fatigue may also be suitable.

While it will be understood that an estimate of a parameter indicative of a degree of muscle fatigue may be determined in many ways, one option is to observe the amplitude of the activity signal and the magnitude of the force with which a wearer performs an exercise. The force may be determined according to F=ma where F is the force in Newtons, m is the mass in kilograms (and may for example be the mass of a weight to be lifted during the exercise, as provided via a user input, or may be biometric data (e.g. information) in the biometric data store, for example where the exercise is a bodyweight exercise and the weight of the wearer is information in the biometric data store), and a is the acceleration in ms⁻¹ (and may be determined from the motion signal, optionally accelerometer signal). If a wearer is performing an exercise in which they are applying the greatest effort they are able to apply, it may be that the force magnitude and the activity signal amplitude will both decrease (in some cases proportionally and/or substantially in parallel) as a function of time as the exercise is performed and the wearer's muscles become fatigued. If the wearer is performing an exercise in which they are applying less than the greatest effort they are apply, it may be that although the exercise may be performed for a longer period, the force magnitude will remain substantially constant (or will decrease more slowly) while the activity signal amplitude will increase as additional motor units are recruited as muscle fibres fatigue to maintain a constant force production.

The controller may be configured to determine an estimate of the resting heart rate of the wearer in dependence on the heart rate signal. The controller may be configured to output the estimate of the resting heart rate of the wearer. The controller may be configured to determine a heart rate zone (e.g. as determined via the Zoldaz method) of the wearer in dependence on the heart rate signal and optionally further in dependence on the resting heart rate of the wearer. The heart rate zone may be determined (e.g. further) in dependence on the maximum heart rate of the wearer. The heart rate zone may be a percentage of the maximum heart rate of the wearer. The controller may be configured to output the determined heart rate zone.

The method may comprise determining an estimate of the resting heart rate of the wearer in dependence on the heart rate signal. The method may comprise outputting the estimate of the resting heart rate of the wearer. The method may comprise determining a heart rate zone (e.g. as determined via the Zoldaz method) of the wearer in dependence on the heart rate signal and optionally further in dependence on the resting heart rate. The method may comprise outputting the determined heart rate zone.

In an example, the controller may be configured to determine an estimate (or the method may comprise estimating) a heart rate zone wherein the heart rate zone is one of several predetermined heart rate zones. The predetermined heart rate zones may comprise a first heart rate zone which is 50% or less of the maximum heart rate of the wearer. The predetermined heart rate zones may comprise a second heart rate zone which is from 51% to 70% of the maximum heart rate of the wearer. The predetermined heart rate zones may comprise a third heart rate zone which is from 71% to 100% (optionally over 100%) of the maximum heart rate of the wearer.

The controller may be configured to determine (or the method may comprise determining) a workout rating in dependence on the heart rate zone and the activity signal. The controller may be configured to output (or the method may comprise outputting) the workout rating. For example, the workout rating may be output to a data store (e.g. a database) The workout rating may be output to a (e.g. the) application, running on a device (e.g. smartwatch, smartphone, tablet, or laptop).

The controller may be configured to perform one or more calibration steps, or the method may comprise one or more calibration steps when the garment is worn by a wearer. For example, the calibration steps may comprise one or more of the following:
the wearer confirming the location of the motion sensor (e.g. the IMU) relative to or on the wearer;
the wearer confirming that they are in a standing position meeting predetermined criteria;
the wearer maintaining the standing position for at least 1 second;
the wearer resting and indicating that they are at rest;
the wearer performing one or more exercises;
receiving a resting activity signal of a detected activity of a muscle from the at least one muscle activity sensor when the muscle is not being activated (e.g. when the muscle (optionally the wearer) is at rest);
receiving a maximum activity signal of a detected activity of the muscle from the at least one muscle activity sensor when the muscle is being maximally activated (e.g. when the wearer indicates that they are activating the muscle as much as they are able to do so);
determining one or more muscle activity zones in dependence on the resting activity signal;
determining one or more muscle activity zones (e.g. further) in dependence on the maximum activity signal;
outputting the one or more muscle activity zones;
determining an estimate of the resting heart rate of the wearer in dependence on the heart rate signal;
outputting the estimate of the resting heart rate of the wearer;
determining an estimate of the maximum heart rate of the wearer in dependence on the heart rate signal;
outputting the estimate of the maximum heart rate of the wearer;
determining one or more heart rate zone in dependence on the estimate of the resting heart rate;
determining one or more heart rate zones in dependence on the estimate of the maximum heart rate;
outputting the one or more heart rate zones;
detecting a parameter indicative of a bioelectrical impedance of the wearer;
outputting the parameter indicative of a bioelectrical impedance of the wearer;
determining a body fat indicator indicative of an amount (e.g. a proportion) of body fat of the wearer (e.g. as a proportion of a total weight of the wearer or in relation to the proportion of the wearer not made up of body fat), optionally an estimate of body fat percentage of the wearer in dependence on the parameter; and/or
outputting the body fat indicator (e.g. the estimate of the body fat percentage of the wearer).

The calibration steps may comprise one or more data input steps. For example, the controller may be configured to prompt the wearer or another user, e.g. a healthcare professional (or the data input steps may comprise prompting the wearer or another user, e.g. a healthcare professional) to input one or more pieces of biometric information, optionally via a (e.g. the) application. The pieces of biometric information may be indicative of one or more of the following:
the age of the wearer;
the gender of the wearer;
the weight of the wearer;
the height of the wearer; and/or
a body fat indicator indicative of an amount (e.g. a proportion) of body fat of the wearer (e.g. as a proportion of a total weight of the wearer or in relation to the proportion of the wearer not made up of body fat), optionally the body fat percentage of the wearer.

Where more than one activity signal is received, the calibration steps may comprise determining the strongest (e.g. highest amplitude) activity signal. Where more than one activity signal is received, the calibration steps may comprise determining the cleanest signal (e.g. the signal having the highest signal to noise ratio). The calibration steps may comprise selecting the anode-cathode pair of electrodes from which the strongest activity signal was received and only receiving activity signals from the said anode-cathode pair subsequently to this selection. The calibration steps may comprise selecting the anode-cathode pair of electrodes from which the cleanest activity signal was received and only receiving activity signals from the said anode-cathode pair subsequently to this selection.

One or more (optionally all) of the calibration steps may performed the first time the garment is worn. One or more (optionally all) of the calibration steps may be performed repeatedly. One or more (optionally all) of the calibration steps may be performed every time the apparatus (e.g. garment) is used (e.g. worn). Some calibration steps may be repeated during a single use. In an example, one or more (optionally all) of the calibration steps may be carried out once per week, optionally once per month, optionally once per three months, optionally once per six months, optionally once per year.

The garment may comprise the controller. The apparatus may comprise a housing configured to retain the controller. The garment may comprise a housing mount. The housing may be configured to be removably mounted to the garment via the housing mount, e.g. such that the controller is in data communication with the garment when the housing is mounted to the garment via the housing mount. The controller may be in data communication with the at least one electrical visual indicator device via the housing mount. The controller may be in data communication with the or each electrode via the housing mount. The controller may be in data communication with the one or more motion sensors via the housing mount. The controller may comprise a wireless communication and power link to the at least one electrical visual indicator device, e.g. via the housing mount. The controller may comprise a wireless communication and power link to the or each electrode, e.g. via the housing mount. The controller may comprise a wireless communication and power link to the one or more motion sensors, e.g. via the housing mount.

In some embodiments, the controller may be a first controller and the apparatus may comprise a second controller. The second controller may be configured to perform any one or more of the functions of the first controller as described hereinbefore. The housing may be a first housing and the apparatus may comprise a second housing configured to retain the second controller. The housing mount may be a first housing mount and the garment may comprise a second housing mount. The second housing may be configured to be removably mounted to the garment via the second housing mount e.g. such that the second controller is in data communication with the second housing mount when the second housing is mounted to the garment via the second housing mount. The second controller may be in data communication with the at least one electrical visual indicator device via the second housing mount. The second controller may be in data communication with the or each electrode via the second housing mount. The second controller may be in data communication with the one or more motion sensors via the second housing mount. The second controller may comprise a wireless communication and power link to the at least one electrical visual indicator device, e.g. via the second housing mount. The second controller may comprise a wireless communication and power link to the or each electrode, e.g. via the housing mount. The second controller may comprise a wireless communication and power link to the one or more motion sensors, e.g. via the housing mount.

Accordingly, the or each controller can be separated from the garment, for example when the garment needs to be washed or a power source of the controller needs to be charged.

The or each housing may be removably mounted to the or each housing mount via a push lock mechanism. In other words, to attach the housing to the housing mount involves a push operation, to push the housing into the housing mount. It may be that the housing and/or the housing mount are configured such that pushing the housing relative to the housing mount causes release of the housing from the housing mount. However, the skilled person will appreciate that there are other ways for the or each housing the be (reversibly) mounted to the or each housing mount.

The apparatus (optionally the controller, the or each housing, or the garment) may comprise one or more input devices. For example, the apparatus (optionally the controller, the or each housing, or the garment) may comprise one or more touch sensors. The apparatus (optionally the controller, the or each housing, or the garment) may comprise one or more buttons. The touch sensor may be a capacitive touch sensor. The touch sensor may be a resistance touch sensor. The touch sensor may be a pressure sensor. The touch sensor may be a temperature sensor.

The controller may be configured to cause (or the method may comprise causing) the brightness of the illumination of the or each electrical visual indicator device to change in dependence on a user inputting an instruction via the input device (e.g. by touching a touch sensor, by depressing a button, etc). For example, the controller may be configured to cause (or the method may comprise causing) the brightness of the illumination of the or each electrical visual indicator device to increase when the touch sensor is touched a first time. The controller may be configured to cause (or the method may comprise causing) the brightness of the illumination of the or each electrical visual indicator device to increase when the touch sensor is touched a second time. The controller may be configured to cause (or the method may comprise causing) the or each electrical visual indicator device to switch off when the touch sensor is touched a third time. The controller may be configured to cause (or the method may comprise causing) the or each electrical visual indicator device to switch back on (optionally wherein the brightness of the illumination of the electrical visual indicator device is a relatively low brightness) when the touch sensor is touched a fourth time.

Alternatively or additionally, the apparatus (optionally the controller, the or each housing, or the garment) may comprise a plurality of input devices (e.g. two or more input devices, optionally three or more input devices). The controller may be configured to adjust (or the method may comprise adjusting) the brightness of the illumination of the or each electrical visual indicator device in dependence on an input from the user made via one or more of the plurality of input devices. For example, the controller may be configured to cause (or the method may comprise causing) the brightness of the illumination of the or each electrical visual indicator device to increase when a first touch sensor is touched. The controller may be configured to cause (or the method may comprise causing) the brightness of the illumination of the or each electrical visual indicator device to decrease when a second touch sensor is touched.

The controller may be configured to select (or the method may comprise selecting) from one or more operation modes in dependence on a user (e.g. the wearer) inputting an instruction via the input device (or via the plurality of input devices) (e.g. by touching a touch sensor, by depressing a button, by inputting instructions via a further device such as a smartwatch, smartphone, tablet, laptop, etc). For example, the controller may be configured to select (or the method may comprise selecting) from one or more of the following operation modes:
a calibration mode wherein one or more calibration steps is carried out;
an exercise mode wherein activity of one or more muscles is detected with at least one muscle activity sensor and optionally wherein at least one electoral visual indicator device outputs a visual indication of the detected activity of the muscle;
a pacing mode wherein the at least one visual indicator outputs a visual indication in an upper pace visual indication mode or a lower pace visual indication mode in dependence on the activity signal;
a rest mode wherein the at least one visual indicator device outputs a rest phase visual indication after an exercise and subsequently stops outputting the rest phase visual indication after a predetermined period;
a body fat determination mode, wherein the controller determines a body fat indicator indicative of an amount (e.g. a proportion) of body fat of the wearer (e.g. as a proportion of a total weight of the wearer or in relation to the proportion of the wearer not made up of body fat), optionally an estimate of the body fat percentage of the wearer in dependence on a parameter detected by a bioelectrical impedance sensor, and outputs the estimate of the body fat indicator (e.g. the body fat percentage) of the wearer;
a performance comparison mode, wherein the at least one visual indicator device outputs a visual indication in dependence on a first (e.g. right-side) activity signal indicative of detected activity of one or more muscles in a first (e.g. right-side) region and a second (e.g. left-side) activity signal indicative of detected activity of one or more muscles in a second (e.g. left-side) region;
a centre of gravity mode wherein the controller determines an estimate of the centre of gravity of the wearer is determined in dependence on one or more received data structures and outputs the estimate of the centre of gravity (optionally further wherein the at least one electrical visual indicator device outputs a visual indication of how far the wearer moves from their centre of gravity); and/or
one or more further modes,
in dependence on a user (e.g. the wearer) inputting an instruction via the input device or via the plurality of input devices (e.g. by touching a touch sensor, by depressing a button, by inputting instructions via a further device such as a smartwatch, smartphone, tablet, laptop, etc).

The apparatus (e.g. garment, optionally the controller housing, optionally the housing mount) may comprise at least one haptic feedback device configured to provide haptic feedback to the wearer. The controller may be configured to cause the haptic feedback device to output haptic feedback. The method may comprise outputting haptic feedback. The method may comprise causing the haptic feedback device to output haptic feedback. The haptic feedback device may be configured to vibrate.

The haptic feedback device may be arranged to cause vibratory motion, when activated, of sufficiently high intensity so as to be felt by the user. The haptic feedback device may be (and/or may be described as) a buzzer.

The controller may be configured to cause (and the method may comprise causing) the haptic feedback device to output haptic feedback in dependence on the apparatus being switched on or off. The controller may be configured to cause (and the method may comprise causing) the haptic feedback device to output haptic feedback in dependence on the apparatus establishing a wireless connection (e.g. a Bluetooth or Wi-Fi connection). The controller may be configured to cause (and the method may comprise causing) the haptic feedback device to output haptic feedback in dependence on an exercise or a series of exercises beginning or ending. The controller may be configured to cause (and the method may comprise causing) the haptic feedback device to output haptic feedback in dependence on a set of repetitions of an exercise being completed. The controller may be configured to cause (and the method may comprise causing) the haptic feedback device to output haptic feedback in dependence on a rest period starting or ending.

Haptic feedback is a convenient way of providing additional information to a user or wearer as they use the apparatus.

The garment may comprise a sustainable textile. For example, the garment may comprise a plant-based textile. The garment may comprise bamboo. The garment may comprise a recycled textile. The garment may comprise a recyclable textile. For example, the garment may comprise recycled and/or recyclable polyester. The garment may comprise elastane. The garment may be a moisture-wicking or sweat-wicking garment. The garment may be a breathable garment. Garments that are moisture-wicking and/or breathable are generally found to be more comfortable when worn, particularly when worn during exercise. It will be understood that while the textile may be a woven textile, this is not required, and the textile may be a non-woven textile.

The garment may comprise (e.g. be) a pair of shorts. The garment may comprise (e.g. be) a pair of leggings. The garment may comprise (e.g. be) a shirt, for example a t-shirt. The garment may comprise (e.g. be) a sock, or a pair of socks. The garment may comprise (e.g. be) a leotard. The garment may comprise welded seams.

The apparatus may comprise a remote device (e.g. a laptop, tablet, smartphone and/or smart watch) comprising a user interface, and computer-readable memory (e.g. a non-transitory computer readable storage medium) storing instructions which, when executed by one or more processors run an application on the remote device.

The application may be configured to prompt a user (e.g. the wearer) to input additional biometric data. The additional biometric data may comprise one or more of: estimated hours of sleep by the wearer (e.g. in minutes within a 24-hour period); estimated caffeine consumption by the wearer (e.g. in milligrams within a 24-hour period); estimated water consumption by the wearer (e.g. in millilitres within a 24-hour period); and indication of the mood of the wearer before performing an exercise; an indication of the mood of the wearer after performing an exercise; an indication of any pain experienced by the wearer; an indication of the level of any pain experienced by the wearer.

The application may be configured to output one or more indicators of the physical health of the wearer in dependence on one or more of: the biometric data; the one or more data structures; the additional biometric data; the or each activity signal; a body fat indicator indicative of an amount (e.g. a proportion) of body fat of the wearer (e.g. as a proportion of a total weight of the wearer or in relation to the proportion of the wearer not made up of body fat); an estimate of the body fat percentage of the wearer; the or each indicator of relative performance; the estimated centre of gravity of the wearer. The application may be configured to output one or more indicators of the mental wellness of the wearer in dependence on one or more of: the biometric data; the one or more data structures; the additional biometric data; the or each activity signal; a body fat indicator indicative of an amount (e.g. a proportion) of body fat of the wearer (e.g. as a proportion of a total weight of the wearer or in relation to the proportion of the wearer not made up of body fat); an estimate of the body fat percentage of the wearer; the or each indicator of relative performance; the estimated centre of gravity of the wearer.

Advantageously, the application can thus be used to provide both qualitative and quantitative feedback to the wearer (or a user) in relation to the wearer's health.

The garment may be worn by a wearer (e.g. a human, in use). The garment may be worn in a gymnasium (e.g. in use). The garment may be worn by a wearer during an exercise. The method my comprise the wearer wearing the garment. The method may comprise the wearer putting on (e.g. donning) the garment. The method may comprise the wearer adjusting the positioning of the garment such that at least one muscle activity sensor for detecting activity of a muscle is at least partially over a muscle. The method may comprise the wearer adjusting the positioning of the garment such that the motion sensor (e.g. IMU) is close to the centre of the lower back. The method may comprise performing one or more exercises.

### DESCRIPTION OF THE DRAWINGS

An example embodiment of the present invention will now be illustrated with reference to the following Figures in which:

FIG. 1A is a plan elevation view diagram, including cut-away sections, of an apparatus according to an example embodiment of the invention wherein the garment is a pair of shorts;

FIG. 1B is a plan elevation view diagram, including cut-away sections, of an apparatus according to a second example embodiment of the invention, wherein the garment is a pair of shorts;

FIG. 2 is a plan elevation view diagram of a locating structure according to an example embodiment of the invention;

FIG. 3 is a flow chart of steps in a method according to an example embodiment of the invention;

FIG. 4 is a flow chart of steps in a method according to an example embodiment of the invention;

FIG. 5 is a flow chart of steps in a method according to an example embodiment of the invention;

FIG. 6 is a flow chart of steps in a method according to an example embodiment of the invention;

FIG. 7 is a flow chart of steps in a method according to an example embodiment of the invention, including the steps of FIG. 6;

FIG. 8 is a flow chart of steps in a method according to an example embodiment of the invention; and

FIG. 9 is a schematic illustration of an apparatus according to an example embodiment of the invention.

### DETAILED DESCRIPTION OF AN EXAMPLE EMBODIMENT

It will be understood by those skilled in the art that any dimensions and relative orientations such as lower and higher, above, and below, and directions such as vertical, horizontal, upper, lower, longitudinal, axial, radial, lateral, circumferential, etc. referred to in this description refer to, and are within expected structural tolerances and limits for, the technical field and apparatus described, and these should be interpreted with this in mind.

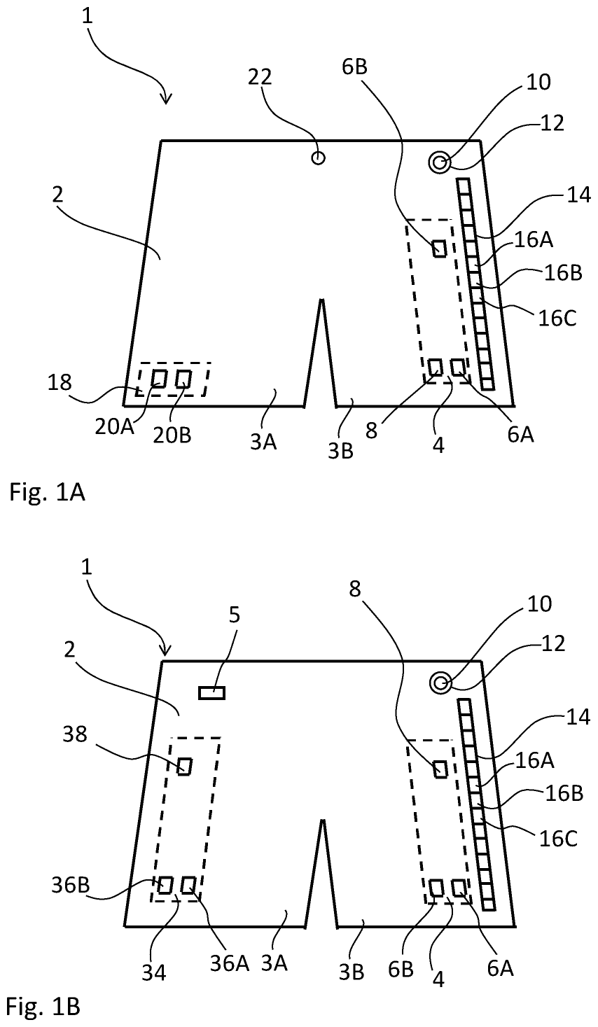

Referring to FIG. 1A, an example embodiment of an apparatus 1 according to the invention comprises a garment 2, here in the form of a pair of shorts. The shorts 2 have two sleeves 3A, 3B, such that the shorts 2 can be worn on the legs of a person.

In this example embodiment, the shorts 2 have an electrical visual indicator device 14, made up of an array of LEDs 16A, 16B, 16C, etc arranged in a line along an external surface of the leftmost sleeve 3B, such that the illumination of the LEDs 16A, 16B, 16C, can be observed when the shorts 2 are worn.

The shorts 2 also have a muscle activity sensor 4 arranged in the leftmost sleeve 3B, for detecting activity of a muscle in the left leg of the wearer. The muscle activity sensor 4 has two measurement electrodes 6A, 6B and a reference electrode 8. The muscle activity sensor 4 is arranged such that each electrode 6A, 6B, 8 is in contact with the skin of the wearer when the shorts 2 are worn (i.e. the electrodes are not typically visible when the shorts 2 are worn).

Furthermore, the shorts 2 have an impedance sensor 18 for detecting bioelectrical impedance of the wearer. The impedance sensor 18 has two electrodes 20A, 20B. The impedance sensor 18 is also arranged such that each electrode 20A, 20B is in contact with the skin of the wearer when the shorts 2 are worn (i.e. the electrodes are not typically visible when the shorts 2 are worn).

The shorts 2 have a motion sensor 22 for detecting motion of the wearer. The motion sensor includes an accelerometer and a gyroscope (not shown) and in some embodiments may include a magnetometer (not shown). Although shown here on the front of the shorts 2, the motion sensor 22 would more typically be positioned at the rear of the shorts 2, such that it is close to the medial part of the lower back when the shorts 2 are worn. In some cases, multiple motion sensors may be provided, for example one on each leg of a pair of shorts, or in other locations. Nevertheless, the skilled person will appreciate that other locations for the motion sensor 22 may be selected in the alternative without departing from the invention. Indeed, in some embodiments the garment 2 may have more than one motion sensor 22.

In this example embodiment, the shorts 2 have a controller 11 (not shown) retained within a controller housing 10. The controller housing 10 is mounted to the shorts 2 via a housing mount 12. When the controller housing 10 is so mounted to the shorts, the controller 11 is in wireless communication with each of the electrical visual indicator device 14, the muscle activity sensor 4, the impedance sensor 18 and the motion sensor 22.

The apparatus also has a computer memory storing a data store, here a database (not shown), storing biometric information about the wearer. The controller 11 is in wireless communication with this computer memory and database.

Accordingly, in use, the controller 11 receives:
activity signals from the muscle activity sensor 4;
bioelectrical impedance signals from the impedance sensor 18; and
motion signals from the motion sensor 22.

In dependence on the activity signals, the controller 11 causes the at least one electrical visual indicator 14 to output a visual indication, here in the form of illuminating one or more of the array of LEDs 16A, 16B, 16C.

In dependence on the bioelectrical impedance signals, the controller 11 determines a body fat indicator indicative of body fat of the wearer, in this case in the form of an estimate of the wearer's body fat percentage. The controller 11 outputs the estimate of the wearer's body fat percentage to the database. The controller 11 then uses the estimate and the biometric information to determine an estimate of the centre of gravity of the wearer.

In some embodiments, the controller may prompt the user to stand in a relaxed position, facing forwards with their feet on the ground and hip-distance apart and to maintain that position for a (e.g. 2 second) period. The controller may estimate a starting position and a starting orientation in dependence on the motion signals during this period.

In dependence on the motion signals and/or the estimated centre of gravity and/or the starting position and/or the starting orientation, the controller 11 determines how far the wearer moves from their centre of gravity and/or a starting position and/or starting orientation (and in which direction) when performing an exercise. If the wearer moves more than a predetermined distance from their centre of gravity (and/or their starting position and/or starting orientation) during an exercise, the controller 11 outputs an alert. In this example embodiment, the alert takes the form of the controller causing the LEDs 16A, 16B, 16C of the electrical visual indicator 14 to flash red (i.e. to output ref light for a period and then to stop outputting red light).

FIG. 1B provides a second example embodiment of an apparatus 1 according to the invention comprises a garment 2, here again in the form of a pair of shorts. Similarly to the example provided in FIG. 1A, this pair of shorts 2 has an electrical visual indicator device 14, made up of an array of LEDs 16A, 16B, 16C, etc arranged in a line along an external surface of one of the sleeves 3B, such that the illumination of the LEDs 16A, 16B, 16C, can be observed when the shorts 2 are worn.

The shorts also 2 have a controller 11, as before, retained within a controller housing 10. The controller housing 10 is mounted to the shorts 2 via a housing mount 12. When the controller housing 10 is so mounted to the shorts, the controller 11 is in electrical communication with each of the electrical visual indicator device 14, the muscle activity sensor 4, the impedance sensor 18 and the motion sensor 22. The apparatus also has a computer memory storing a database (not shown) storing biometric information about the wearer. The controller 11 is in wireless communication with this computer memory and database.

In this instance, the shorts 2 also have two muscle activity sensors 4, 34, in the leftmost 3B and rightmost 3A sleeves of the shorts 2, respectively. Accordingly, the two muscle activity sensors 4, 34 are respectively arranged to detect activity of a muscle in the right leg, and the in left leg, of the wearer. The muscle activity sensors 4, 34 each have two measurement electrodes 6A, 6B, 36A, 36B and a reference electrode 8, 38. The muscle activity sensors 4, 34 are arranged such that each electrode 6A, 6B, 8, 36A, 36B, 38 is in contact with the skin of the wearer when the shorts 2 are worn (i.e. the electrodes are not typically visible when the shorts 2 are worn).

The shorts also have a haptic feedback device 5, arranged to cause vibratory motion, when activated, of sufficiently high intensity so as to be felt by the user. The haptic feedback device in this instance may be (and/or may be described as) a buzzer,

Accordingly, in use, the controller 11 receives activity signals from the two muscle activity sensors 4, 34. In dependence on the activity signals from the left activity sensor 4, the controller 11 determines a degree of activity of the muscle in the left leg of the wearer. In dependence on the activity signals of the right activity sensor 34, the controller 11 determines a degree of activity of the muscle in the right leg of the wearer. The controller 11 then compares the degree of activity of the two muscles, and in dependence on this comparison, causes the electrical visual indicator 14 to output a visual indication of relative muscle performance.

In this example embodiment, if the degree of muscle activity of the muscle in the left leg is determined to be greater than that of the muscle in the right leg, the LEDs 16A, 16B, 16C illuminate in green (e.g. the controller may cause the LEDs 16A, 16B, 16C to illuminate in green, e.g. to output green light). If the degree of muscle activity of the muscle in the right leg is determined to be greater than that of the muscle in the left leg, the LEDs 16A, 16B, 16C illuminate in blue (e.g. the controller may cause the LEDs 16A, 16B, 16C to illuminate in blue, e.g. to output blue light). While in this example embodiment, the shorts 2 have an electrical visual indicator device 14, made up of an array of LEDs 16A, 16B, 16C, it will be understood that in some embodiments each LED may be an electrical visual indicator device. Accordingly, it may be that each LED 16A, 16B, 16C (and optionally further LEDs) illuminate together. However, in the alternative, it may be that each LED 16A, 16B, 16 (and optionally further LEDs) illuminates independently of each other LED.

In addition, the controller 11 is in wireless communication with the haptic feedback device 5. The controller 11 causes the haptic feedback device 5 to output haptic feedback by buzzing in response to various events. In this example embodiment, if the activity signal from the activity sensor 4, 34 in one sleeve 3A, 3B is indicative of muscle activity, while the activity signal from the activity sensor 4, 34 in the other sleeve 3A, 3B is indicative or no muscle activity, the controller may cause the haptic feedback device 5 to buzz. This will then alert the user to the fact that something is wrong, for example that the electrodes of one activity sensor 4, 34 are no longer contacting their skin.

The shorts 2 in this example are fabricated from moisture-wicking recycled polyester-based textile, with welded seams. The controller housing 10 and mount 12 are fabricated from high density polyethylene. The electrodes 6A, 6B, 8, 36A, 36B, 38 are dry, passive electrodes. The LEDs 16A, 16B, 16C are flexible LEDs. Communication between the controller 11 and the muscle activity sensors 4, 34, the electrical visual indictor 14, the motion sensor 22, and the haptic feedback device 5, takes place via printed electronics in each of these devices and Bluetooth™ connections. The textile of the shorts 2 according to this example embodiment contains no wires or conductive threads. However, in some example embodiments wire, conductive threads, and/or conductive ink may be used in one or more circuits.

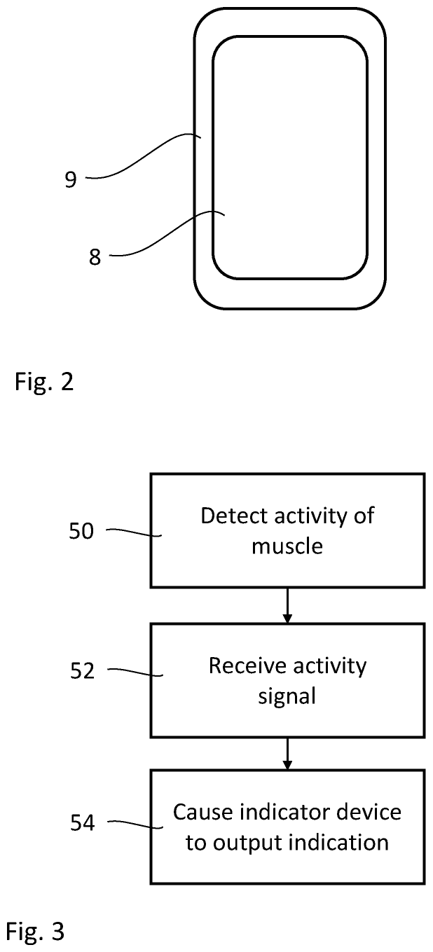

FIG. 2 is a plan view diagram of a locating structure 9 surrounding an electrode 6A, 6B, 8 36A, 36B, 38. In embodiments, one, some, or each electrode of the garment 2 may be provided with such a locating structure. The locating structure 9 has an outermost surface which increases the friction between the garment 2 and the wearer's skin when the garment 2 is worn. This decreases the extent to which the electrodes 6A, 6B, 8, 36A, 36B, 38 move relative to the wearer's skin. In this example embodiment, the locating structure is fabricated from silicone. However, it will be appreciated that other materials (in particular solid elastomers, e.g. latex) may be equally suitable.

Although the example embodiment of the invention according to FIG. 1A does not include a second muscle activity sensor 34, the skilled person will appreciate that in some embodiments this may be included. In some embodiments, the garment 2 may have two or more muscle activity sensors, and each while each muscle activity sensor may be arranged to detect activity of a different muscle, this is not required. In some cases more than one muscle activity sensor may be arranged to detect activity of the same muscle.

Although the example embodiment of the invention according to FIG. 1A does not include a haptic feedback sensor 5, the skilled person will appreciate that in some embodiments this may be included. Indeed, substantially any feature of the embodiment of FIG. 1A may be a feature of the embodiment of FIG. 1B, and vice-versa.

Although in the embodiments of FIGS. 1A and 1B the electrical visual indicator device 14 is made up of an array of LEDs 16A, 16B, 16C arranged along a line, this is not required. For example, other devices may be used to output visual indications. Furthermore, light emitters other than LEDs may be used. The light emitters need not be arranged along a line, and other patterns or arrangements may be used without departing from the invention. In some embodiments, the garment 2 may have more than one electrical visual indicator 14, for example, where the garment is a pair of shorts as in FIGS. 1A and 1B, the garment may have an electrical visual indicator 14 on each sleeve 3A, 3B. The garment may have more than one electrical visual indicators 14 on each sleeve. Similarly, while in the embodiments of FIGS. 1A and 1B the controller 11 may be considered part of the shorts 2, this is not required, and it may be the case that the controller 11 is provided separately from and/or remote from the garment 2.

Furthermore, while in the embodiments of FIGS. 1A and 1B the garment 2 is a pair of shorts 2, this is also not required. The garment may be substantially any garment, provided at least a portion of the garment is configured to stay at least partially in contact with the wearer's skin, at least partially over a muscle, when the garment is worn. For example, the garment may be a t-shirt, a pair of leggings, a sock, a leotard, etc.

It will be understood that the visual indications described above are examples only.

While it may be that the controller 11 outputs an alert by causing the LEDs 16A, 16B, 16C to flash red if the wearer moves more than a predetermined distance from their centre of gravity during exercise, other visual alerts may be output in the alternative. For example, the controller may cause the LEDs 16A, 16B, 16C to illuminate in red (e.g. to output red light) (or any other colour) with or without flashing, or for some but not all of the LEDs 16A, 16B, 16C to illuminate (e.g. output light), or for the LEDs 16A, 16B, 16C to be switched off. Furthermore, the alert need not be a visual alert, and may be an audio alert, or may take the form of causing the haptic feedback device 5 to output haptic feedback. Where the alert is a visual alert, it need not be output via the electrical visual indicator, and may for example be output to a different device, such as a wearer's smart watch.

FIG. 3 is a flow chart of steps in a method according to an example embodiment of the invention. In this embodiment, the method includes: detecting 50 activity of a muscle (e.g. a muscle at least partially covered by a muscle activity sensor 4, 34); receiving 52 an activity signal (e.g. from a muscle activity sensor 4, 34); and causing 54 an electrical visual indicator device 14 to output a visual indication.

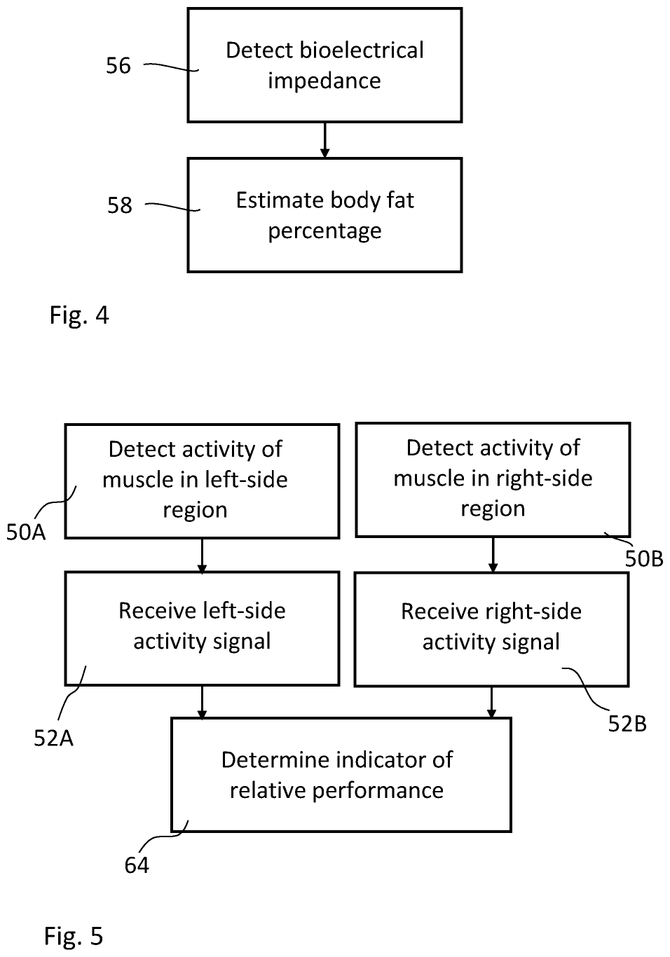

FIG. 4 is a flow chart of steps in a method according to an example embodiment of the invention. In this embodiment, the method includes: detecting 56 bioelectrical impedance of a wearer of the garment 2; and estimating 54 the body fat percentage of the wearer of the garment 2. In some embodiments, the method may also include outputting the estimate of body fat percentage, for example to a database.

FIG. 5 is a flow chart of steps in a method according to an example embodiment of the invention. In this embodiment, the method includes: detecting 50A activity of a muscle in a left-side region of a wearer of the garment 2 (e.g. a muscle at least partially covered by a muscle activity sensor 4); receiving 52A a left-side activity signal (e.g. from a muscle activity sensor 4 arranged to detect activity of a muscle in a right-side region of the wearer); detecting 50B activity of a muscle in a right-side region of a wearer of the garment 2 (e.g. a muscle at least partially covered by a muscle activity sensor 34); receiving 52B a right-side activity signal (e.g. from a muscle activity sensor 34 arranged to detect activity of a muscle in a left-side region of the wearer); and determining 64 an indicator of relative performance of the muscle in the left-side region and the muscle in the right-side region (e.g. in dependence on the left-side activity signal and the right-side activity signal).

In some embodiments, the method may also include outputting the indicator of relative performance, for example to a database. The method may also include causing the electrical visual indicator device 14 to output a visual indication in dependence on the indicator of relative performance.

Although the detection 50A, 50B of activity of the muscles in the left- and right-side regions occurs substantially simultaneously in this example embodiment, this is not required. It may be the case that detection of activity of the muscle in the left-side region 50A occurs before detection of activity of the muscle in the right-side region 50B, or vice-versa. Similarly, although the left- and right-side activity signals are received 52A, 52B substantially simultaneously in this example embodiment, this is also not required. It may be the case that the left-side activity signal is received 52A before the right-side activity signal is received 52B, or vice-versa.

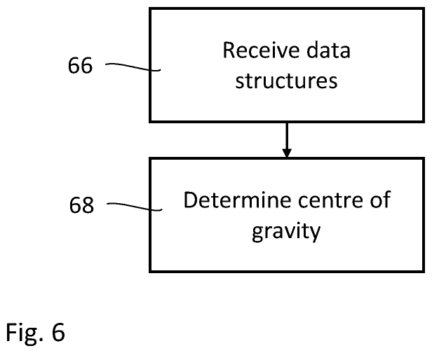

FIG. 6 is a flow chart of steps in a method according to an example embodiment of the invention. In this embodiment, the method includes: receiving 66 data structures (e.g. from a database); and determining 68 an estimate of the centre of gravity of the wearer in dependence on the received data structures. In some example embodiments, the method may also include outputting the estimate of the centre of gravity, for example to a database.

In this example embodiment, the data structures include: an estimate of the total weight of the wearer; an estimate of the body fat percentage of the wearer (this may be obtained via the method of FIG. 4); an estimate of the width of the feet of the wearer (e.g. an average distance, orthogonal to the length of the feet of the wearer (from a heel region to a toe region), at the widest part of the soles of the feet of the wearer); and a separation distance (e.g. a distance between the outermost lateral point of the wearer's left foot and the outermost lateral point of the wearer's right foot when the wearer positions their feet at a known separation).

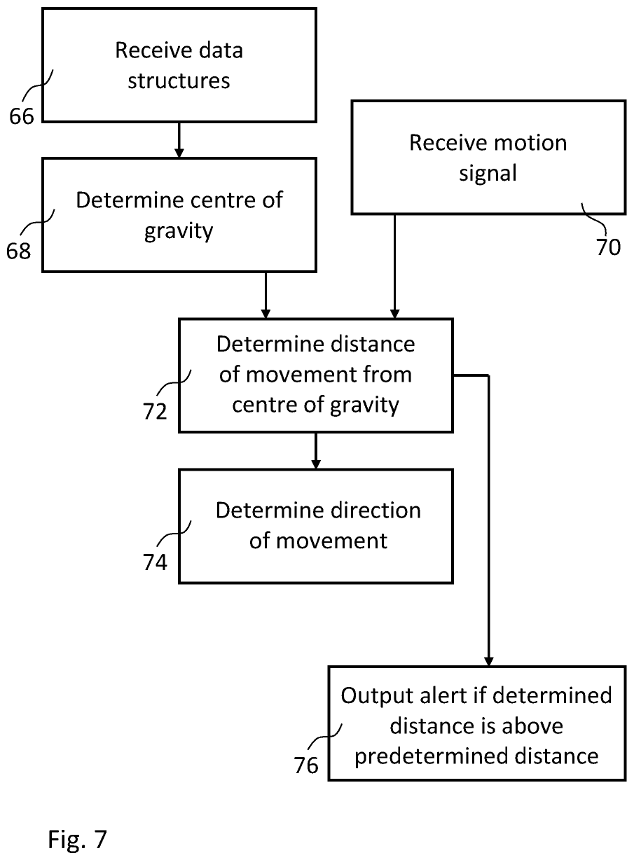

FIG. 7 is a flow chart of steps in a method according to an example embodiment of the invention, including some of the steps of the method of FIG. 6. In this embodiment, the method includes: receiving 66 data structures (e.g. from a database); determining 68 an estimate of the centre of gravity of the wearer in dependence on the received data structures; receiving 70 a motion signal (e.g. from a motion sensor 22); determining 72 an estimated distance of movement of the wearer from the estimated centre of gravity (e.g. when the wearer performs an exercise) in dependence on the estimated centre of gravity and the motion signal; determining 74 a direction of movement of the wearer from the estimated centre of gravity (e.g. when the wearer performs an exercise) in dependence on the estimated centre of gravity and the motion signal; and outputting 76 an alert if the estimated distance from the centre of gravity is above a predetermined distance. The alert may be indicative of the determined direction of the movement of the wearer from the estimated centre of gravity.

The step of outputting 74 the alert may be optional. Where the method includes the step of outputting 74 the alert, the alert may be in one of several forms. For example, the alert may be an audio alert, a visual alert, a haptic alert, etc.

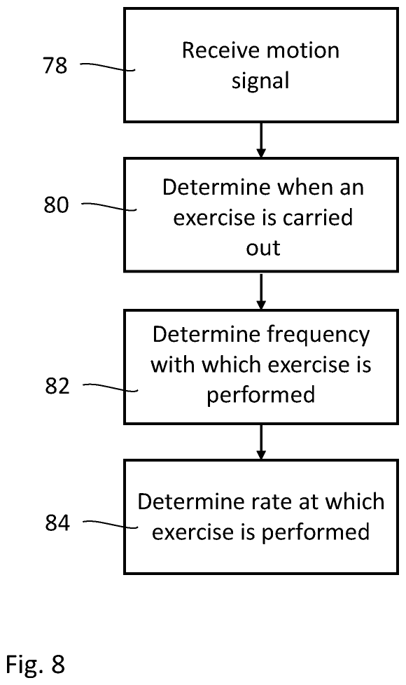

FIG. 8 is a flow chart of steps in a method according to an example embodiment of the invention. In this embodiment, the method includes: receiving 78 a motion signal; determining 80 when an exercise is carried out, in dependence on the received motion signal; determining 82 an estimate of the frequency with which the exercise is performed, in dependence on the received motion signal; and determining 84 an estimate of the rate at which the exercise is performed, in dependence on the estimated frequency and the received motion signal.

It will be understood that steps in each example embodiment method may be performed in the order set out hereinbefore. However, in some example embodiments, the steps may be performed in other orders and/or some steps may be performed simultaneously to other steps.

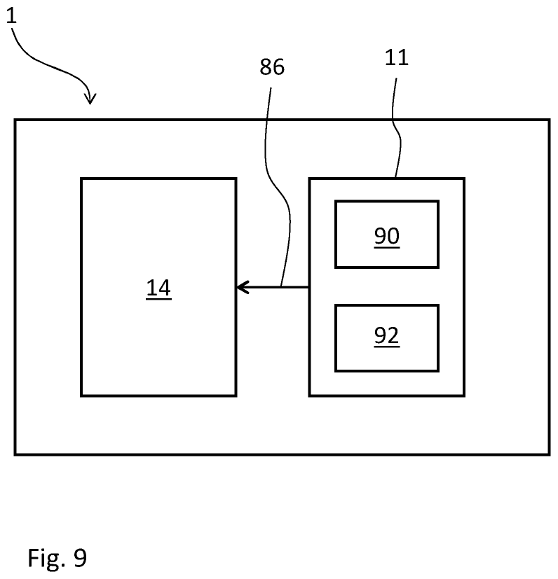

FIG. 9 is a schematic illustration of an apparatus 1 according to an example embodiment of the invention. The apparatus 1 has at least one electrical visual indicator device 14 and a controller 11. The controller 11 is configured to send signals 86 to the electrical visual indicator device 14. The controller 11 is also typically configured to transmit data elsewhere, for example to further components of the apparatus 1, and/or to devices external to the apparatus 1, via a wireless data connection. The signals 86 include signals generated by the controller 11 in dependence on data received by the controller 11, for example from user inputs and/or from the muscle activity sensor(s) 4, 34, motion sensor 22, bioelectrical impedance sensor 18, etc. The controller 11 in this example is realised by one or more processors 90 and a computer-readable memory 92. The memory 92 stores instructions which, when executed by the one or more processors 90, cause the apparatus 1 to operate as described herein.

Although the controller 11 is shown as being part of the apparatus 1, it will be understood that one or more components of the controller 11, or even the whole controller 11, can be provided separate from the apparatus 1. For example, the controller may be remote from the apparatus 1 and may exchange signals with the electrical visual indicator 14 by wireless communication.

In summary, there is provided an apparatus (1) comprising a garment (2), the garment comprising: at least one muscle activity sensor (4, 34) for detecting activity of a muscle at least partially covered by the garment; at least one electrical visual indicator device (14); and a controller (11) configured to: receive an activity signal indicative of detected activity of the muscle from the at least one muscle activity sensor; and cause the at least one electrical visual indicator device to output a visual indication of the detected activity of the muscle in dependence on the received activity signal.

Throughout the description and claims of this specification, the words “comprise” and “contain” and variations of them mean “including but not limited to”, and they are not intended to and do not exclude other components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment, or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

What is claimed is:

1. An apparatus comprising:
a garment comprising:
at least one muscle activity sensor configured to detect activity of a muscle at least partially covered by the garment; and
at least one electrical visual indicator device; and
a controller, the controller being configured to:
receive from the at least one muscle activity sensor an activity signal indicative of detected activity of the muscle; and
cause the at least one electrical visual indicator device to output a visual indication of the detected activity of the muscle in dependence on the received activity signal.

2. (canceled)

3. The apparatus according to claim 1, wherein the at least one electrical visual indicator device comprises a plurality of LEDs and wherein:
the controller is configured to cause one or more of the plurality of LEDs to illuminate in dependence on the received activity signal.

4. The apparatus according to claim 1, wherein the at least one muscle activity sensor is a plurality of muscle activity sensors, each muscle activity sensor configured to detect activity of a muscle at least partially covered by the garment, in a respective region of the body of the wearer.

5. The apparatus according to claim 4, wherein the controller is configured to:
receive a respective activity signal indicative of detected activity of a muscle from each respective muscle activity sensor; and
cause the at least one electrical visual indicator device to output a visual indication of the detected activity of the muscle in the respective region of the body of the wearer, in dependence on each respective activity signal.

6. The apparatus according to claim 3, wherein the plurality of LEDs are arranged in one or more lines.

7. The apparatus according to claim 1, wherein:
the visual indication comprises at least two visual indication modes, each visual indication mode being a different visual indication mode to each other visual indication mode of the at least two visual indication modes;
the at least one electrical visual indicator device is configured to output visual indications in the at least two visual indication modes; and
the controller is configured to cause the at least one electrical visual indicator device to output a visual indication in one of the at least two visual indication modes, in dependence on the received activity signal.

8. The apparatus according to claim 7, wherein the controller is configured to cause the at least one electrical visual indicator device to:
output a visual indication in a first visual indication mode if the activity signal is indicative of a first degree of activity of the muscle; and
output a visual indication in a second visual indication mode, different to the first visual indication mode, if the activity signal is indicative of a second degree of activity of the muscle.

9. The apparatus according to claim 7, wherein the controller is configured to:
determine a frequency with which the activity signal is indicative of activation of a muscle; and
cause the at least one electrical visual indicator device to output a visual indication in a lower pace indication mode if the frequency is below a predetermined threshold frequency or to output a visual indication in an upper pace visual indication mode if the frequency is above a predetermined threshold frequency.

10. The apparatus according to claim 1, wherein the apparatus comprises a computer readable memory comprising exercise information about one or more exercises, and wherein the controller is configured to:
receive exercise information from the computer readable memory;
determine when the wearer is performing an exercise in dependence on the received exercise information and the activity signal;
determine when the wearer has completed the exercise in dependence on the received exercise information and the activity signal;
cause the at least one electrical visual indicator device to output a rest phase visual indication when the exercise has been completed; and
subsequently, cause the at least one electrical visual indicator device to stop outputting the rest phase visual indication after a predetermined period.

11. The apparatus according to claim 1, wherein the apparatus comprises at least one impedance sensor configured for detecting a parameter indicative of a bioelectrical impedance of the wearer, the at least one impedance sensor comprising at least two electrodes,
and wherein the controller is configured to:
receive the detected parameter;
determine a body fat indicator in dependence on the detected parameter, the body fat indicator being indicative of an amount of body fat of the wearer, and
output the body fat indicator.

12-13. (canceled)

14. The apparatus according to claim 1, wherein
the muscle activity sensor comprises:
at least two first electrodes arranged to contact the skin of a first region of a wearer of the garment and to thereby detect muscle activity of one or more muscles in the first region;
at least two second electrodes arranged to contact the skin of a second region of the wearer and to thereby detect muscle activity of one or more muscles in the second region,
and wherein the controller is configured to:
receive a first activity signal indicative of detected activity of one or more muscles in the first region from the first electrodes;
receive a second activity signal indicative of detected activity of one or more muscles in the second region from the second electrodes;
determine one or more indicators of relative performance of the one or more muscles in the first region and the one or more muscles in the second region, in dependence on the first activity signal and the second activity signal; and
output the one or more indicators of relative performance,
optionally wherein the first electrodes comprise right-side electrodes arranged to contact the skin of a right-side region of the wearer of the garment and to thereby detect muscle activity of one or more muscles in the right-side region, and wherein the second electrodes comprise left-side electrodes arranged to contact the skin of a left-side region of the wearer of the garment and to thereby detect muscle activity in one or more muscles in the left-side region.

15-17. (canceled)

18. The apparatus according to claim 1, wherein the garment comprises a locating structure for each electrode, the or each locating structure comprising an outermost surface configured to increase the frictional forces between the garment and the skin of the wearer of the garment to thereby limit the movement of each respective electrode relative to the skin of the wearer,
optionally wherein the locating structure is defined on an inner surface of the garment, the inner surface configured to be in contact with the skin of the wearer when the garment is worn by the wearer, optionally wherein the locating structure comprises an elastomer material, further optionally, wherein the garment is at least partially fabricated from a first textile having a first elasticity and the garment comprises a compression layer at least partially covering the first textile, wherein the compression layer has a second elasticity greater than the first elasticity.

19-20. (canceled)

21. The apparatus according to claim 1, wherein the controller is configured to receive an estimate of the centre of gravity of the wearer.

22. The apparatus according to claim 1, the apparatus further comprising a computer readable memory comprising one or more data structures together indicative of:
an estimate of the total weight of the wearer;
a body fat indicator indicative of an amount of body fat of the wearer;
an estimate of a width of the feet of the wearer; and
a predetermined separation distance,
the apparatus further comprising a controller configured to:
receive one or more of the one or more data structures from the biometric data store;
determine an estimate of the centre of gravity of the wearer in dependence on the one or more received data structures; and
output the estimate of the centre of gravity.

23-24. (canceled)

25. The apparatus according to claim 21, wherein the garment comprises at least one motion sensor configured for detecting motion of the wearer, and wherein the controller is configured to:
receive a motion signal indicative of motion of the wearer from the at least one motion sensor;
determine a distance, in a direction, from the estimate of the centre of gravity that the wearer moves when performing the exercise, in dependence on the motion signal; and
output the distance from the centre of gravity.

26. The apparatus according to claim 25, wherein the controller is configured to output an alert if the wearer moves more than a predetermined distance from the centre of gravity.

27. The apparatus according to claim 25, wherein the controller is configured to:
determine when an exercise has been carried out in dependence on the motion signal;
determine the frequency with which the exercise is carried out in a period and thereby determine an estimate of the rate at which the exercise is carried out; and
output the estimated rate at which the exercise is carried out.

28. The apparatus according to claim 1, wherein;
the garment comprises at least one motion sensor configured for detecting motions of the wearer; and
the controller is configured to:
receive a motion signal indicative of the motions of the wearer from the at least one motion sensor;
estimate a starting orientation and optionally a starting position in dependence on the motion signal when the wearer is at rest; and
estimate a change in orientation from the starting orientation and optionally estimate a distance from the starting position in dependence on the motion signal when the wearer performs an exercise.

29. (canceled)

30. The apparatus according to claim 1, comprising a housing configured to retain the controller and wherein the garment comprises a housing mount, wherein the housing is configured to be removably mounted to the garment via the housing mount, and/or
wherein the garment comprises at least one haptic feedback device configured to provide haptic feedback to the wearer, and
the controller is configured to cause the haptic feedback device to output haptic feedback.

31. (canceled)

32. A method of visually indicating activity of at least one muscle, the method comprising:
providing an apparatus comprising a garment, the garment comprising:
at least one muscle activity sensor configured to detect activity of a muscle at least partially covered by the garment; and
at least one electrical visual indicator device,
the method comprising:
receiving an activity signal indicative of a detected activity of the muscle from the at least one muscle activity sensor; and
causing the at least one electrical visual indicator device to output a visual indication of the activity of the muscle in dependence on the received activity signal.
